(19) United States
(12) Patent Application Publication
(10) Pub. No.: US 2018/0280302 A1
(43) Pub. Date: Oct. 4, 2018

(54) SOLID DISPERSIONS OF COMPOUNDS USING POLYVINYL ALCOHOL AS A CARRIER POLYMER

(71) Applicant: MERCK PATENT GMBH, Darmstadt (DE)

(72) Inventors: Shawn KUCERA, Cedar Park, TX (US); Dieter LUBDA, Bensheim (DE); Dave MILLER, Georgetown, TX (US); Chris BROUGH, Georgetown, TX (US)

(73) Assignee: MERCK PATENT GMBH, Darmstadt (DE)

(21) Appl. No.: 15/545,141

(22) PCT Filed: Dec. 22, 2015
(86) PCT No.: PCT/EP2015/002596

§ 371 Date: Jul. 20, 2017

Related U.S. Application Data
(60) Provisional application No. 62/105,380, filed on Jan. 20, 2015.

(51) Int. Cl. A61K 9/14; A61K 47/32; A61K 31/496; A61K 31/4422; A61K 31/192

(52) U.S. Cl. CPC A61K 9/146; A61K 47/32; A61K 31/192; A61K 31/4422; A61K 31/496

(57) ABSTRACT

The present invention refers to a method for producing storage-stable solid dispersions of poorly soluble pharmaceutically active compounds comprising polyvinyl alcohol as carrier matrix. The invention also refers to the prepared compositions and their use.

The present invention refers storage-stable solid dispersions of poorly soluble pharmaceutically active compounds comprising polyvinyl alcohol as carrier matrix. The invention also refers to these compositions and their use.

### TECHNICAL FIELD

In order to increase the rate of dissolution it is well known to prepare formulations of poorly soluble compounds in form of solid dispersions. These solid dispersions can be created by a number of methods, including, but not limited to, spray-drying, melt extrusion, and thermoki-netic compounding.

Solid dispersions are defined as being a dispersion of one or more active ingredients in an inert solid matrix and can broadly classified as those containing a drug substance in the crystalline state or in the amorphous state [Chiou W. L., Riegelman S. Pharmaceutical applications of Solid dispersion systems; J. Pharm Sci. 1971, 60 (9), 1281-1301]. Solid dispersions containing pharmaceutical active ingredients in the crystalline state provide dissolution enhancement by simply decreasing surface tension, reducing agglomeration, and improving wettability of the active substance [Sinswat P., et al.; Stabilizer choice for rapid dissolving high potency itraconazole particles formed by avaporative precipitation into aqueous solution; Int. J. of Pharmaceutics, (2005) 302; 113-124]. While crystalline systems are more thermodyna-mically stable than their amorphous counterparts, the crystalline structure must be interrupted during the dissolution process, requiring energy. Solid dispersions containing an active ingredient, this means a drug, dissolved at the molecular level, known as amorphous solid solutions, can result in a significant increase in dissolution rate and extent of supersaturation [DiNunzio J. C. et al. III Amorphous compositions using concentration enhancing polymers for improved bioavailability of itraconazole; Molecular Pharmaceutics (2008); 5(6):968-980]. While these systems have several advantages, physical instability can be problematic due to molecular mobility and the tendency of the drug to recrystallize. Polymeric carriers with high glass transition temperatures seem to be well suited to stabilize these systems by limiting molecular mobility.

Various processes can be used to create solid dispersions.

In general, these systems can be produced by processes either utilizing solvents or which require the melting of one or more of the added substances. Techniques that utilize solvents to form amorphous solid solutions include solvent evaporation [Chowdary K. P. R., Suresh Babu K. V. V.; Dissolution, bioavailability and ulcerogenic studies on solid dispersions of indomethacin in water-soluble cellulose polymers. Drug Dev. Ind. Pharm. (1994); 20(5):799-813.], co-precipitation [Martínez-Ohárriz M. C. et al.; Solid dispersions of diflunisal-PVP: polymorphic and amorphous states of the drug.; Drug Dev Ind Pharm. (2002); 28(6):717-725], freeze drying [Sekikawa H. Et al.; Dissolution behavior and gastrointestinal absorption of dicumarol from solid dispersion systems of dicumarol-polyvinylpyrrolidine and dicumarol-beta-cyclodextrin. Chem Pharm Bull (Tokyo). (1983), 31(4):1350-1356], supercritical fluid technologies (Rogers T. L.; Johnston K. P., Williams R. O.; III Solution-based particle formation of pharmaceutical powders by supercritical or compressed fluid CO2 and cryogenic spray-freezing technologies. Drug Dev Ind Pharm. (2001), 27(10):1003-1015), and spray drying [Friesen D T, Shanker R, Crew M, Smithey D T, Curatolo W J, Nightingale J A. Hydroxypropyl methylcellulose acetate succinate-based spray-dried dispersions: an overview. Molecular Pharmaceutics. (2008), 5(6), 1003-1019]. To achieve an amorphous dispersion through spray drying, for example, the solvent or co-solvent system utilized must be suitable to dissolve both the polymeric carrier vehicle and the compound of interest. In summary, these methods require the use of a solvent system, often organic in nature, to dissolve an inert carrier and active drug substance (Serajuddin A. T. M.; Solid dispersion of poorly water-soluble drugs: early promises, subsequent problems, and recent breakthroughs. J Pharm Sci. (1999), 88(10), 1058-1066). Once a solution is formed, the solvent is subsequently removed by a mass transfer mechanism dependent on the manufacturing technique chosen. Although solvent-based techniques such as spray drying are relatively common, they suffer from several disadvantages. Selection of a solvent system that is compatible with the active substance and carrier polymer may prove to be difficult or require very large amounts of organic solvent. This presents a safety hazard at the manufacturing facility as organic solvents must be collected and disposed of properly to limit the environmental impact (Lakshman J. P., Cao Y., Kowalski J., Serajuddin A. T. M.; Application of melt extrusion in the development of a physically and chemically stable high-energy amorphous solid dispersion of a poorly water-soluble drug. Molecular Pharmaceutics. (2008), 5(6), 994-1002). Furthermore, organic solvents may be difficult to fully remove from processed materials. This solvent removal step may require prolonged times at elevated temperatures, presenting an additional cost to the manufacturer. For these reasons, fusion processing has gained increased acceptance over solvent-based techniques and has become the method of choice for the large-scale manufacture of amorphous solid solutions (Leuner C.; Dressman J.; Improving drug solubility for oral delivery using solid dispersions; Eur J Pharm Biopharm. (2000), 50, 47-60).

Although hot melt extrusion, a fusion processing technique, has been used in the food and plastics industry for more than a century, it has only recently gained acceptance in the pharmaceutical industry for the preparation of these systems.

In this method, a thermoplastic carrier is combined with a pharmaceutical active substance and optional inert excipients and further additives. For an amorphous dispersion via melt extrusion, the polymeric carrier vehicle must first possess a thermoplasticity that allows the polymer to be passed through the extruder and also must be thermally stable at barrel temperatures above the glass transition temperature or melting point of the polymer.

The mixture is introduced into rotating screws that convey the powder into a heated zone where shear forces are imparted into the mixture, compounding the materials until a molten mass is achieved. While this manufacturing method has many advantages over solvent-based methods, it does have significant limitations. During processing, drug substances are exposed to elevated temperatures for prolonged periods of time. Although a variety of factors can affect the residence time distribution of an extruded substance, these times typically fall within the 1- to 2-min range but have also been reported to be as long as 10 min (Breitenbach J., Melt extrusion: from process to drug delivery technology. Eur J Pharm Biopharm. (2002), 54, 107-117). This prolonged exposure to elevated temperatures can induce decomposition of thermally labile compounds or accelerate decomposition of chemically unstable compounds. When these processing issues are encountered, the addition of processing aids such as plasticizers may allow processing to be carried out at a lower temperature (Schilling S. U. et al.; Citric acid as a solid-state plasticizer for Eudragit RS PO; J Pharm Pharmacol. (2007), 59(11), 1493-1500). However, the addition of a plasticizer can affect the solid-state physical stability of the solid dispersion once formed. That is, the increased molecular mobility may allow the drug substance to transition to the more thermodynamically stable state when the glass transition temperature of the resulting amorphous solid solution is at least 50° C. higher than the storage temperature (Hancock B. C., Shamblin S. L., Zografi G.; Molecular mobility of amorphous pharmaceutical solids below their glass transition temperatures; Pharm. Res. (1995) 12(6), 799-806).

In this context, polyvinyl alcohol (PVA) seems to be an excellent compound. Polyvinyl alcohol (PVA) is a synthetic water-soluble polymer that possesses excellent film-forming, adhesive, and emulsifying properties. It is prepared from polyvinyl acetate, where the functional acetate groups are either partially or completely hydrolyzed to alcohol functional groups. As the degree of hydrolysis increases, the solubility of the polymer in aqueous media increases, but also crystallinity and melting temperature of the polymer increase. In addition to this, the glass transition temperature varies depending on its degree of hydrolysis. For example, a 38% hydrolyzed material has no melting point, but a glass transition temperature of approximately 48° C., whereas a 75% hydrolyzed material has a melting temperature of approximately 178° C., an 88% hydrolyzed material has a melting point of approximately 196° C., and a 99% material has a melting point of approximately 220° C., but the polymer tends to degrade rapidly above a temperature of 200° C.

Polyvinyl alcohol is soluble in water, but almost insoluble in almost all organic solvents, excluding, in some cases, ethanol. This aspect of the polymer makes it very difficult to form amorphous and solid dispersions through spray drying when the drug has a limited solubility in aqueous media.

Likewise, the polymer is impossible to extrude via melt extrusion because either the temperatures required are too high to prevent degradation or the polymer does not flow well in the melt extruder barrel.

But U.S. Pat. No. 8,236,328 describes a pharmaceutical composition comprising a dispersion comprising a low-solubility drug and a matrix combined with a concentration-enhancing polymer. At least a major portion of the drug is amorphous in the dispersion. The compositions improve the stability of the drug in the dispersion, and/or the concentration of drug in a use environment. PVA is claimed as a matrix material and a concentration enhancing polymer and the drug is substantially amorphous. In this case, PVA and the API are dissolved in a 4/1 methanol/water cosolvent system and then spray dried. This formulation system showed not any benefit, as described by the dissolution AUC, when compared to the control (undispersed amorphous) drug.

U.S. Pat. No. 5,456,923 A provides a process for producing a solid dispersion, which overcomes disadvantages of the conventional production technology for solid dispersions. The invention comprises employing a twin-screw extruder in the production of a solid dispersion. In accordance with the invention, a solid dispersion can be expediently produced without heating a drug and a polymer up to or beyond their melting points and without using an organic solvent for dissolving both components and the resulting solid dispersion has excellent performance characteristics. The process claims a polymer that is natural or synthetic and can be employed as a raw material where the polymer's functions are not adversely affected by passage through the twin screw extruder. Although PVA is claimed as a viable polymer, the extrusion of pharmaceutically acceptable PVA in a binary mixture with an API is impossible without exceeding the melting point of the polymer, which would damage the functionality of the polymer.

EP 2 105 130 A1 describes a pharmaceutical formulation comprising a solid dispersion having an active substance embedded in a polymer in amorphous form, and an external polymer as a recrystallization inhibitor independently of the solid dispersion. The external polymer is claimed as a solution stabilizer. The active substance should be sparingly soluble or less sparingly soluble in water. PVA is claimed as a polymer to form the solid dispersion. It is claimed that the solid dispersion is obtained by melt extrusion. The process comprises melting and mixing the polymer and the active ingredient, cooling, grinding, mixing with the external polymer, and producing a pharmaceutical formulation. It is claimed that the melting is carried out at a temperature below the melting point of the drug. It is also claimed that the melting is carried out at a temperature above the Tg or melting point of the polymer, but from 0.1-5° C. below the melting point of the API. The melting point of pharmaceutical grades of PVA is normally above 178° C., although the glass transition temperature is in the range of 40-45° C. But for a skilled person it is clear that this invention can be processed according to the invention only in a few exceptions because suitable conditions can only be set with a few special active ingredients and it would be difficult to process a binary mixture of PVA and a poorly soluble API according to what is disclosed here.

WO 2010/032958 A discloses an amorphous solid dispersion comprising adefovir dipivoxil, a water soluble polymer and a sugar alcohol. PVA is claimed as one of the polymer substances, either neat or in a mixture. A method is described, wherein a water-soluble polymer substance and an API are dissolved in an organic solvent and the solution is allowed to be adsorbed to a sugar alcohol or dispersed therein. In one embodiment of this invention spray drying is carried out.

WO 2013/012959 A discusses a compound of a defined structure in a solid matrix polymer. The polymer is soluble in an aqueous solution, water, or an aqueous solution of pH 5.0 or higher. Polyvinyl alcohol (PVA) is one of the applied polymers. The disclosed solid dispersion can comprise one or more excipients. A recrystallization inhibitor can also be added to the system, preferably Poloxamer 188. The active compound should be amorphous. In the process as disclosed a solid dispersion is prepared by forming a solution of the active compound, the solid matrix, and a solvent and then removing the solvent. The solvent can be neat or a co-solvent system, which may comprise water. After mixing the solvent can be removed by flash freezing followed by freezing, flash freezing followed by drying in a centrifugal concentrator, or by spray drying.

KR 2013-0028824 A discusses a solid dispersion of tacrolimus and a method to prepare it. The method includes melting the tacrolimus, a polymer melt base, and a surfactant to prepare the melt mixture, solidifying the melt by cooling, and then pulverizing the mixture. PVA is disclosed as a polymer melt base. Processing conditions are in the range of 80-150° C. Processing is carried out by melting the components in a glass beaker while stirring. HPMC (Hydroxypropylmethylcellulose) and Gelucire® (a mixture of glycerides and esters of polyethylene glycol) are taught as polymer melt bases in the patent examples. Although PVA is listed as an acceptable polymer, the processing conditions of 80-150° C. are not acceptable to melt pharmaceutical grades of PVA by the method taught in the patent. PVA 4-75 has a Tₘ of around 180° C. and PVA 4-88 has a Tₘ of around 200° C.

CN 103040725 A discusses a method of grinding or milling drospirenone with hydrophilic non-polymer excipients or water soluble excipients to create a solid dispersion. The milled/ground material is then screened (sized). PVA is cited as an example of a water soluble excipient. Grinding by mortar and pestle or ball mill are methods of manufacturing. But it was found that this method is not suitable to homogeneously disperse the API within the polymer matrix.

### Problem to be Solved

Because of the above stated physical and chemical properties of polyvinyl alcohol (PVA), it is extremely difficult to formulate a solid dispersion, especially when the compound of interest to be included has a poor solubility in aqueous media, wherein a solid dispersion could be manufactured via spray-drying. Also, because the polymer cannot be melt extruded without addition of a significant amount of additives, a solid dispersion by melt extrusion can only be made with great difficulties. Therefore, it is an object of the present invention to provide uniformly dispersed active ingredients in PVA in amorphous form. It is also an object of this invention to provide these compositions in a storage stable form.

### SUMMARY OF THE INVENTION

The object of the present invention is a pharmaceutical composition comprising polyvinyl alcohol (PVA) as functional excipient in combination with at least one poorly soluble pharmaceutical active ingredient (API), which is produced in a method wherein the substances submitted are thoroughly compounded in a thermokinetic mixer for less than 300 seconds, preferably for a duration time between 5 and 180 seconds, more preferably between 7 to 60 seconds, but most preferably between 10 to 30 seconds to minimize the heat exposure of compounded materials,

whereby the temperature in the chamber of the thermokinetic mixer is raised to 100 to 200° C. by rotational shear and friction energy, preferably to a temperature in the range of 100-150° C., in particular to a temperature in the range of 100-130° C., and
whereby the pharmaceutically active ingredient(s), the functional excipient and the processing agent(s) optionally submitted form a melt blended pharmaceutical composition.

Compositions according to the present invention comprise pharmaceutically acceptable PVA having a degree of hydrolysis in the range of greater than 72.2% but less than 90% according to the requirements of the European Pharmacopoeia or between 85-89% according to the United Stated Pharmacopoeia, and a molecular weight in the range of 14 000 g/mol to 250 000 g/mol.

The poorly soluble pharmaceutical active ingredients of these compositions are biologically active agents in form of a weak base, a weak acid or a neutral molecule. The comprising pharmaceutically acceptable PVA of is composed of one or more grades of PVA of differing molecular weights and of differing grades of hydrolysis.

In compositions according to the present invention the comprising pharmaceutically acceptable PVA may be combined with another excipient. In special cases PVA as functional excipient can be combined with another pharmaceutically acceptable polymer.

In general compositions according to the present invention comprise a week base as biologically active agent and PVA in a ratio in the range of 1:99 to 1:1 by weight, preferably the ratio of active agent to PVA is in the range 1:70 to 1:2.

In a special embodiment of the invention the composition is produced with at least one active agent, which is ground or pre-milled to mean particle sizes in the range of 1 to 1000 μm, preferably to mean particle sizes in the range of 1 μm to 100 μm, most preferably in the range of 10 μm to 100 μm, before it is processed. Compositions disclosed here comprise the pharmaceutical active ingredient(s) in an amorphous nano-crystalline or micro-crystalline form.

Surprisingly, in compositions of the invention the pharmaceutical active ingredient is dissolved upon dissolution by a factor of at least 1.2 higher compared to the thermodynamic solubility of said ingredient alone in the polymer matrix. Moreover the comprising PVA is crystalline, semi-crystalline or amorphous after processing.

The method disclosed here is particularly suited to dissolute poorly soluble pharmaceutical active ingredients as biologically active agents in form of a weak base, a weak acid or a neutral molecule in PVA. These poorly soluble pharmaceuticals as active ingredient may be selected from the group itraconazole, ibuprofen and nifedipine.

It is possible to apply pharmaceutically acceptable PVA, which is composed of one or more grades of PVA of differing molecular weights and of differing grades of hydrolysis. Furthermore, this excipient may be combined with another excipient. This means, that the method may be carried out with PVA, which is combined with another pharmaceutically acceptable polymer as a further excipient or carrier.

To implement the method according to the invention a week base as biologically active agent and PVA are submitted in the thermokinetic mixer in the correct amounts and in a ratio in the range of 1:99 to 1:1 by weight, preferably the ratio of active agent to PVA is in the range 1:70 to 1:2. Then the temperature in the chamber of the thermokinetic mixer is raised to 100 to 200° C. by rotational shear and friction energy. Preferably, the mixing is carried out at lower temperatures and the temperature in maintained in the range of to 100-150°, preferably to a temperature in the range of 100-130° C.

Particularly good mixing resulted when the particle size of the poorly soluble active ingredient used was set in advance to a average diameter in the range of 1 to 1000 μm, preferably to mean particle sizes in the range of 1 μm to 100 μm, most preferably in the range of 10 μm to 100 μm. To achieve this, the active agent is ground or milled to the desired mean particle sizes.

The performed experiments have shown that thermokinetic processing according to the present invention may be performed for a duration time between 5 and 120 seconds, preferably between 7 and 180 seconds, more preferably between 7 to 60 seconds, but most preferably between 10 to 30 seconds to minimize the heat exposure of compounded materials.

Thus, a composition is prepared by thermokinetic compounding comprising one or more pharmaceutical active ingredient(s), which is (are) homogeneously dispersed in a polyvinyl alcohol matrix. This composition comprises the pharmaceutical active ingredient(s) in an amorphous nano-crystalline or micro-crystalline form. As mentioned above, surprisingly, compositions are prepared in which the pharmaceutical active ingredient, upon dissolution, is dissolved by a factor of at least 1.2 higher compared to the thermodynamic solubility of said ingredient alone in the polymer matrix.

Compositions according to the invention comprise PVA as an excipient in crystalline, semi-crystalline or amorphous form after processing.

In addition to the compositions themselves also oral dosage forms comprising these compositions are subject matter of the present invention. Such dosage forms may be prepared in form of tablets, as beads, granules, pellets, capsules, suspensions, emulsions, gels and films.

### DETAILED DESCRIPTION

While the making and using of various embodiments of the present invention are discussed in detail below, it should be appreciated that the present invention provides more applicable inventive concepts than described here in detail. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “thermokinetic compounding” or “TKC” refers to a method of thermokinetic mixing until melt blended. TKC may also be described as a thermokinetic mixing process in which processing ends at a point sometime prior to agglomeration. A detailed description of this process can be found in U.S. Pat. No. 8,486,423 B2.

As used herein, the phrase “a homogenous, heterogenous, or heterogeneously homogenous composite or an amorphous composite” refers to the various compositions that can be made using the TKC method.

As used herein, the term “heterogeneously homogeneous composite” refers to a material composition having at least two different materials that are evenly and uniformly distributed throughout the volume.

As used herein, the term “thermokinetic chamber” refers to an enclosed vessel or chamber in which the TKC method is used to make the novel compositions of the present invention. In a TKC chamber the average temperature inside the chamber is ramped up to a pre-defined final temperature over the duration of processing to achieve thermokinetic compounding of the one or more APIs and the one or more pharmaceutically acceptable excipients into a composite.

As used herein, “bioavailability” is a term meaning the degree to which a drug becomes available to the target tissue after being administered to the body. Poor bioavailability is a significant problem encountered in the development of pharmaceutical compositions, particularly those containing an active ingredient that is not highly soluble.

As used herein, the phrase “pharmaceutically acceptable” refers to molecular entities, compositions, materials, excipients, carriers, and the like that do not produce an allergic or similar untoward reaction when administered to humans in general.

As used herein, “pharmaceutically acceptable carrier” or “pharmaceutically acceptable materials” includes any and all solvents, dispersion media, coatings, antibacterial and antifungal agents, isotonic and absorption delaying agents, and the like. The use of such media and agents for pharmaceutical active substances is well known in the art.

The API (active pharmaceutical ingredient) may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base.

As used herein, “poorly soluble” refers to having a solubility such that the dose to be administered cannot be dissolved in 250 ml of aqueous media ranging in pH from 1 to 7.5, drugs with slow dissolution rates, and drugs with low equilibrium solubilities, for example resulting in decreased bioavailability or reduced pharmacological effect of the therapeutic agent being delivered.

For the characterizing particle sizes “mean” or “average particle sizes” (d₅₀) are given. These value are assessed by means of sieve analysis using wire mesh plates. Sieve plates are arranged from largest aperture to smallest, and the particle size is assessed based on proportion of material caught at each level. All sieve plates have apertures with square cross sections. The mean particle size (D₅₀) is then calculated from the sieving results. D₅₀ is defined as the equivalent diameter where 50% of the mass (of particles) of the sampled powder has a smaller diameter, and thus 50% of the material remains coarser. D₅₀ can therefore be described as the average particle size.

As used herein, “derivative” refers to chemically modified inhibitors or stimulators that still retain the desired effect or property of the original API. Such derivatives may be derived by the addition, removal, or substitution of one or more chemical moieties on the parent molecule. Such moieties may include, but are not limited to, an element such as a hydrogen or a halide, or a molecular group such as a methyl group. Such a derivative may be prepared by any method known to those of skill in the art. The properties of such derivatives may be assayed for their desired properties by any means known to those of skill in the art. As used herein, “analogs” include structural equivalents or mimetics.

A variety of administration routes are available for delivering the APIs to a patient in need. The particular route selected will depend upon the particular drug selected, the weight and age of the patient, and the dosage required for therapeutic effect. The pharmaceutical compositions may conveniently be presented in unit dosage form. The APIs suitable for use in accordance with the present disclosure, and their pharmaceutically acceptable salts, derivatives, analogs, prodrugs, and solvates thereof, can be administered alone, but will generally be administered in admixture with a suitable pharmaceutical excipient, diluent, or carrier selected with regard to the intended route of administration and standard pharmaceutical practice.

The APIs may be used in a variety of application modalities, including oral delivery as tablets, capsules or suspensions; pulmonary and nasal delivery; topical delivery as emulsions, ointments or creams; transdermal delivery; and parenteral delivery as suspensions, microemulsions or depot. As used herein, the term “parenteral” includes subcutaneous, intravenous, intramuscular, or infusion routes of administration.

The solution agent used in the solution can be an aqueous such as water, one or more organic solvents, or a combination thereof. When used, the organic solvents can be water miscible or non-water miscible. Suitable organic solvents include but are not limited to ethanol, methanol, tetrahydrofuran, acetonitrile, acetone, tert-butyl alcohol, dimethyl sulfoxide, N,N-dimethyl formamide, diethyl ether, methylene chloride, ethyl acetate, isopropyl acetate, butyl acetate, propyl acetate, toluene, hexanes, heptane, pentane, and combinations thereof.

The excipients and adjuvants that may be used in the presently disclosed compositions and composites, while potentially having some activity in their own right, for example, antioxidants, are generally defined for this application as compounds that enhance the efficiency and/or efficacy of the effective ingredients. It is also possible to have more than one effective ingredient in a given solution, so that the particles formed contain more than one effective ingredient.

As stated, excipients and adjuvants may be used to enhance the efficacy and efficiency of the APIs.

Thermal binders may also be used in the presently disclosed compositions and composites.

Depending on the desired administration form the formulations can be designed to be suitable in different release models, which are well known to the skilled person, as there are: immediate, rapid or extended release, delayed release or for controlled release, slow release dosage form or mixed release, including two or more release profiles for one or more active pharmaceutical ingredients, timed release dosage form, targeted release dosage form, pulsatile release dosage form, or other release forms.

The resulting composites or compositions disclosed herein may also be formulated to exhibit enhanced dissolution rate of a formulated poorly water soluble drug.

The United States Pharmacopeia-National Formulary mandates that an acceptable polyvinyl alcohol for use in pharmaceutical dosage forms must have a percentage of hydrolysis between 85 and 89%, as well as a degree of polymerization between 500 and 5000. The degree of polymerization (DM) is calculated by the equation:

DM=(Molar Mass)/((86)−(0.42(the degree of hydrolysis)))

The European Pharmacopoeia mandates that an acceptable polyvinyl alcohol for use in pharmaceutical dosage forms must have an ester value no greater than 280 and a mean relative molecular mass between 20,000 and 150,000. The percentage of hydrolysis (H) can be calculated from the following equation:

H=((100−(0.1535)(EV))/(100−(0.0749)(EV)))×100

Where EV is the ester value of the polymer. Thus, only polymers with a percentage of hydrolysis greater than 72.2% are acceptable according to the European Pharmacopoeia monograph.

Due to the fact that polyvinyl alcohol is a non-thermoplastic polymer, it is unsuitable to be processed via traditional melting methods (hot melt extrusion) to formulate a solid dispersion, although there are several variations of this mixing procedure.

Recently a new mixing method has been developed, wherein the influence of thermal and kinetic energy are combined and at the same time the mixture is carried out by shear stress.

U.S. Pat. No. 8,486,423 A describes this thermokinetic compounding method (mixing method) to enhance the solubility of poorly soluble active pharmaceutical ingredients. The work discloses the use of thermokinetic processing of compounds in blends of poorly soluble active pharmaceutical ingredients with pharmaceutically acceptable polymers, such as cellulose derivatives, acrylic derivatives, and polyvinyl derivatives. The patent also discloses that pharmaceutically acceptable grades of polyvinyl alcohol (PVA) may be a suitable matrix polymer.

This thermokinetic compounding, also known to the expert as Kinetisol® method, has been shown to yield results comparable to melt extrusion. The advantage of the thermokinetic compounding method is that for high glass transition temperature polymers, no additional plasticizer is needed to process the polymers.

Thermokinetic compounding, as described in U.S. Pat. No. 8,486,423 A, offers numerous advantages, such as brief processing times, low processing temperatures, high shear rates, and the ability to compound thermally incompatible materials into more homogeneous composites. The method requires no organic or aqueous solvents to dissolve the pharmaceutical carrier and the API and plasticizers are not required to enhance the melt flow properties of the polymeric carrier.

One example of a thermokinetic compounder, as described in U.S. Pat. No. 8,486,423 has a high horsepower motor driving the rotation of a horizontal shaft with teeth-like protrusions that extend outward normal to the rotational axis of the shaft. The portion of the shaft containing the protrusions is contained within a second enclosed vessel where the compounding operation takes place, i.e., a thermokinetic chamber. The high rotational velocity of the shaft coupled with the design of the shaft protrusions imparts kinetic energy onto the materials being processed. The compounder is operated by a digital control system which allows the operating parameters, i.e., revolutions per minute and ejection temperature, to be set prior to the compounding operation. A temperature analyzer measures the average temperature inside the compounder. The machine can be run in automatic mode in which the digital control system ejects the material once the set temperature is reached within the vessel.

Although polyvinyl alcohol cannot be processed in the known hot melt extrusion processes, it was found that the new thermokinitic compounding process (TKC process) is suitable to manufacture a homogenously dispersed solid solution of pharmaceutical active ingredient in polyvinyl alcohol. In particular, poorly soluble pharmaceutical active ingredient can be homogeneously mixed with PVA to build a solid dispersion. Furthermore, it was found by experiments that PVA in the different degrees of hydrolysis can be homogeneously mixed by the TKC process with poorly soluble active ingredients, especially PVA that is in accordance with the European Pharmacopoeia monograph and which is a pharmaceutically acceptable PVA with hydrolysis grades between 72.2% and 90%, and especially which includes grades of PVA that are pharmaceutically acceptable by either the USP (hydrolysis between 85-89%) or Ph.Eur. (hydrolysis greater than 72.2%, but less than 90%). These PVA qualities have a molecular weight in the range of 14,000 g/mol to 250,000 g/mol.

According to the present invention it is possible to process compositions of biologically active ingredient comprising one or more grades of PVA of differing molecular weights in the range of 14,000 g/mol to 250,000 g/mol, or compositions of a biologically active ingredient comprising one or more grades of PVA with differing degrees of hydrolysis.

Compositions according to the invention may comprise a biologically active ingredient combined with a PVA that is pharmaceutically acceptable, which is combined with another pharmaceutically acceptable polymer. Such pharmaceutically acceptable polymer can also be selected from the group of hydrophilic polymers and can be a primary or secondary polymeric carrier that can be included in the composition disclosed herein include polyethylene-polypropylene glycol (e.g. POLOXAMER™), carbomer, polycarbophil, or chitosan. Hydrophilic polymers for use with the present invention may also include one or more of hydroxypropyl methylcellulose, carboxymethylcellulose, hydroxypropyl cellulose, hydroxyethyl cellulose, methylcellulose, natural gums such as gum guar, gum acacia, gum tragacanth, or gum xanthan, and povidone. Hydrophilic polymers also include polyethylene oxide, sodium carboxymethycellulose, hydroxyethyl methyl cellulose, hydroxymethyl cellulose, carboxypolymethylene, polyethylene glycol, alginic acid, gelatin, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

In another embodiment of the invention a composition of a biologically active ingredient can be combined with a PVA that is pharmaceutically acceptable and with one or more pharmaceutically acceptable excipient(s). Such an excipient may have a limited miscibility with the biologically active ingredient and may be a polymeric or non-polymeric excipient. Suitable excipients may be selected from the group excipients consisting of lactose, glucose, starch, crystalline cellulose, simple syrup, glucose solution, starch solution, gelatin solution, carboxymethyl cellulose, methyl cellulose, dried starch, sodium alginate, powdered agar, calcium carmelose, a mixture of starch and lactose, sucrose, glycerin and starch, lactose, sucrose esters, cyclodextrins, cellulose derivatives and combinations thereof, and/or selected from the group excipients consisting of calcium carbonate, kaoline, silicic acid, bentonite, colloidal silicic acid, talc, and combinations thereof, and/or selected from the group consisting of phosphatidyl choline derivatives, butter, hydrogenated oil, a mixture of a quarternary ammonium base and sodium lauryl sulfate, dipalmitoyl phosphadityl choline, deoxycholic acid and salts, sodium fusidate, stearates, sorbitan esters, polyoxyethylene sorbitan fatty acid esters, sodium lauryl sulfate, oleic acid, lauric acid, vitamin E TPGS, and combinations thereof, and or selected from the group consisting of glycolic acid, salts of glycolic acid, or combinations thereof. This means the one or more excipients are selected from the group consisting of a pharmaceutically acceptable polymer, a thermolabile polymeric excipient, and a non-polymeric excipient.

In a preferred embodiment of the invention a composition of a biologically active ingredient can be combined with a PVA that is pharmaceutically acceptable and with one or more pharmaceutically acceptable excipients wherein the one or more excipients are selected from the group consisting of starch, crystalline cellulose, starch solution, carboxymethyl cellulose, shellac, methyl cellulose, polyvinyl pyrrolidone, dried starch, calcium carmelose, polyethylene glycol, polyoxyethylene sorbitan fatty acid esters, polyoxyethylene alkyl ethers, poloxamers (polyethylene-polypropylene glycol block copolymers), polyoxyethylated glycolysed glycerides, polyethylene glycols, polyglycolyzed glycerides, polyacrylates, polymethacrylates, polyvinylpyrrolidones, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

The present invention also includes compositions in which the biologically active ingredient is present in the composition in an amorphous, nanocrystalline, or microcrystalline form.

Due to the particular conditions during the thermokinetic compounding method, it is possible to prepare formulations of active compounds containing higher concentrations than can be produced in conventional processes. Especially compositions are subject of the invention in which the biologically active ingredient is dissolved by a factor of at least 1.2 higher compared to the thermodynamic solubility of the biologically active ingredient alone.

From compositions according to the invention oral dosage forms in the form of a tablet, beads, granules, capsule, etc. may be prepared. These dosage forms may comprise the applied PVA in crystalline, semi-crystalline, or amorphous form after processing depending on the applied hydrolysation grade of the PVA and depending on the dosage form.

Although it is in general a problem to incorporate different PVAs by hot melt extruding methods regardless of degree of hydrolysis, the new thermokinitic compounding process (TKC process) makes it possible to use PVA in a solid dispersion comprising an API that is a biologically active agent which is poorly soluble in aqueous media. In this context, in particular the so-called Kinetisol®) process has been found suitable to allow the use of different PVAs in this physical form. The biologically active agent (API) can be a weak base, a neutral molecule or a weak acid. Particularly preferred the active ingredient can be itraconazole, ibuprofen or nifedipine.

The TKC process allows it to incorporate PVA in low but also quite high concentrations in the compositions. Thus, in formulations including a week base as biologically active agent (API) the ratio of active agent to PVA may be in the range of 1:99 to 1:1 by weight. Preferably the ratio of active agent to PVA is in the range 1:70 to 1:2.

Accordingly, for example, 1 g of a poorly soluble drug and 99 g of PVA are just as mixed as 33.3 g of the same active ingredient with 66, 7 g of PVA on laboratory scale.

But not only weak bases as active ingredients can be mixed with PVA in this way and ratio but also neutral molecules or a weak acids can be treated in the same manner and are applicable with PVA in the same rations by weight.

The Kinetisol®) process provides a solution to the production of oral drug delivery formulations based on a commercial plastics compounding process, which is developed into a cGMP-compliant pharmaceutical operation. This process allows the pharmaceutical manufacturing at a commercial scale.

To carry out the Kinetisol®) process the composition is loaded into the processing chamber of the dispersing machine at room temperature where a computer control module is utilized to set the desired rotational processing speed and ejection set point (Dispersol Technologies LLC (Austin, Tex., USA). As the blades rotate at high speeds, heat is generated through shear and friction within the chamber. Rotational speeds and temperatures inside the processing chamber are monitored and recorded in real time by the computer control module and are detailed in subsequent sections. Compositions of the present invention are processed for a few seconds at temperatures in the range of 100 to 220° C., but temperatures in the range of 100-185° C. were sufficient for an intensive compounding. Very homogeneous dispersions are already achieved, at processing temperatures in the range of 100-150° and the experiments have shown that at temperatures in the range of 100-130° the dispersion of API in the PVA matrix is satisfying, so that it is possible to compound the active ingredient at low temperatures and to avoid degradation.

After reaching the predetermined processing temperature, the compounder ejects the molten material directly into liquid nitrogen to rapidly quench the material. The compounded material is placed under vacuum for about 30 minutes to prevent moisture adsorption. Subsequently, the resulting mixture may be milled. On laboratory scale, for example, a Laboratory L1 A Fitzmill (Fitzpatrick Inc., Elmhurst) may be used for this milling step. This mill is equipped with a 0.0020 in. screen in a knives forward configuration, operating at 9,000 rpm.

The experiments carried out have shown that PVA with a suitable degree of hydrolysis can be perfectly mixed with poorly soluble drugs by the described Kinetisol®) process.

Since a low water solubility of an active ingredient in general accompanies a low bioavailability after its administration in a pharmaceutical preparation, the prepared systems of the invention also contribute to improving the bioavailability of sparingly water-soluble, ingredients, regardless of whether it is weakly basic, weakly acidic or neutral.

Examples of such pharmaceutically active ingredients that are weak bases include, Acetriptan (pKa of 4.9) acyclovir, Amitriptyline, Amlodipine, Atenolol, Atropine, Ciprofloxacin, Diazepam, Dilitiazem, Diphenhydramine, Diphenhydramine HCl, Epinephrine, Ephedrine, Glucosamine, Glucosamine sulfate, Hydrochlorothiazide, Imatinib, Loratadine, Metoprolol, Nelfinavir, Nevirapine (pKₐ 2.8), Nortriptyline, Phenytoin, Propoxyphene, Propranolol [Propranolol HCl ((±)-1-isopropylamino-3-(1-naphthyloxy) propan-2-ol hydrochloride)], Prednisolone, Reserpine, Terfenadine, Tetracycline, Theophylline, Pergolide, Pseudoephedrine, Vardenafil hydrochloride or Verapamil hydrochloride and mixtures thereof.

Examples of pharmaceutically active ingredients that are weak acids include, captopril, diclofenac, enalapril, furosemide, ketoprofen, phenobarbital, naproxen, ibuprofen, lovstatin, penicillin G, piroxicam and ranitidine.

Weak acids and weak bases and their properties are discussed in detail in Physical Pharmacy. 4^{th} Edition, ed. Alfred Martin, Lippincott Williams & Wilkins, 1993, Chapter 7.

Examples of pharmaceutically active ingredients that are neutral include Tetracyclines, Penicillins or Sulfonamide.

Advantageously the active ingredients and PVA as carrier can be mixed by the TKC process without the addition of any solvent or additive known to the skilled person at a temperature below the melting point of the active ingredient but also the melting point of the carrier must not be exceeded.

The applied TKC process is flexible and can blend said materials with or without agglomeration (rendering the polymer molten) with varying degrees of effect. This flexibility is particularly useful when additives are needed for special reasons. The term thermokinetic compounding refers to thermokinetic mixing used for melt blending. The main advantage of this process in comparison to HME processes is that the materials are exposed to heat for very short durations, and yet completely different substances can be connected to each other in a mixture. While one of these substances may be non-melting the other substance can become plastic. The result is that the different compounds are not simply mixed but rather the two substances become bonded without degrading the more heat sensitive substance. This means the processing time is brief and the heat exposure of the materials is minimized.

Compositions according to the present invention, comprising PVA of different degrees of hydrolysis may be processed only for a few seconds. In order to achieve a very fast and good distribution of the poorly soluble drug in the PVA matrix, it may be useful to grind crystalline substances in addition before carrying out the thermokinetic compounding. Depending on the added ingredients of the composition the mixing time can vary. For compositions at all scales the mixing lasts only for a few seconds. The advantage of such a reduction of the thermokinetic processing durations is a substantial reduction of possibly occurring decomposition of active ingredients (APIs) but also of the excipient or carrier, here of PVA. This advantage is important for thermally labile APIs, which typically undergo significant degradation during thermal processing, as well as APIs that are subject to oxidation.

Depending on the nature of the active ingredient it shows amorphous, crystalline, or intermediate morphology after thermokinetic compounding.

In order to achieve a homogeneous distribution of the active substance in the excipient or carrier in a short process time it may be helpful, as already said, to grind or mill the active substance to average particle sizes in the range of 1 to 1000 μm. This applies especially if the API is crystalline. Thus the mean particle size of the API bulk material can be set by dry milling in the range of 1 μm to 100 μm, preferably in the range of 10 μm to 100 μm.

Suitable methods to reduce or set the particle size of the active substance are:
dry milling of crystalline API to reduce the particle size of the bulk material.
wet milling of crystalline API with a pharmaceutically acceptable solvent to reduce the particle size of the bulk material.
melt milling of a crystalline API with one or more molten pharmaceutical excipients having limited miscibility with the crystalline API to reduce the particle size of the bulk material.
milling crystalline API in the presence of polymeric or non-polymeric excipient to create ordered mixtures where fine drug particles adhere to the surface of excipient particles and/or excipient particles adhere to the surface of fine drug particles.

As already mentioned above, thermokinetic processing may be performed for less than 5, 10, 15, 20, 25, 30, 35, 40, 45, 50, 55, 60, 75, 100, 120, 150, 180, 240 and 300 seconds. Generally, thermokinetic processing may be performed for between 5 and 120 seconds, preferably between 7 and 180 seconds, more preferably between 7 to 60 seconds, but most preferably between 10 to 30 seconds to minimize the heat exposure of compounded materials. With the thermokinetic compounding the polymeric material, here PVA is rendered molten through mechanical generation of kinetic energy, not by the addition of external heat, and therefore molten processing can be achieved below the T_{g} of the polymeric material. Thus the process offers the same advantages of hot melt extrusion, like non-solvent processing, providing intimate mixing of materials in the molten state and highly efficient, scalable manufacturing.

Through the experiments, which are described in the following, it was found, that by the thermokinetic compounding amorphous solid dispersion systems of PVA and poorly soluble APIs are produced without the use of processing agents like plasticizers or thermal lubricants. Nevertheless stable solid dispersion formulations are produced and formulations with drug release characteristics which are not influenced by additives. Therefore, by preparing amorphous solid dispersion systems of PVA and poorly soluble APIs by the TKC process at temperatures below the melting points and glass transition temperatures of both improved dispersion systems can be produced, which are suitable, optionally after further process steps, for tableting, encapsulation and other pharmaceutically acceptable dosage form development techniques known to those skilled in the art, e.g., injection molding, compression molding, film pressing, pelletizing, hot melt extrusion, melt granulation, tablet compression, capsule filling, and film-coating.

### Examples

Even without any further explanations, it is assumed that a person skilled in the art can make use of the above description in its widest scope. The preferred embodiments and examples are therefore to be regarded merely as descriptive but in no way limiting disclosures.

For better understanding and for illustration, examples are given below which are within the scope of protection of the present invention. These examples also serve for the illustration of possible variants.

The complete disclosure of all applications, patents and publications mentioned above and below are incorporated by reference in the present application and shall serve in cases of doubt for clarification.

It goes without saying that, both in the examples given and also in the remainder of the description, the quoted percentage data of the components present in the compositions always add up to a total of 100% and not more. Given temperatures are measured in ° C.

### Itraconazole (ITZ) Compositions

### Processing

Itraconazole, PVA, and PVP K25 (when used) (Table 1) were first blended in a powder mixer to achieve blend uniformity. The resulting mixture was then dosed into the Kinetisol® compounder and processed according to the conditions found in Table 1. Upon reaching the pre-set ejection temperature, the material was ejected from the compounder, pressed into a disc, and allowed to cool to room temperature. Following quenching, the composition was milled using a Fitzpatrick L1A FitzMill. The mill was operated in hammer forward orientation, fitted with a 500 μm screen at an operating RPM of 5,000. The particles which passed through a 250 μm sieve were selected for further testing.

### Characterization

ITZ-PVA KinetiSol® processed samples and the corresponding controls were analyzed for crystalline character by x-ray diffraction (XRD) using an Equinox 100 benchtop x-ray diffractometer (Inel, Inc., Stratham, N.H.). Samples were placed in an aluminum crucible and loaded into a rotating sample holder. Samples were analyzed for 600 seconds using a Cu K radiation source (λ=1.5418 Å) operating at 42 kV and 0.81 mA in a 2-theta range of 0-150°. Results are reported from 10-35° (2-theta) as this is the primary region for the diffraction of x-rays by ITZ.

### Dissolution

All dissolution tests were conducted in a Vankel™ VK-7000 (Varian, Inc.) USP apparatus II (paddle) dissolution tester. The dissolution medium was maintained at 37° C. with a recirculating heater and a paddle speed of 75 RPM was constant throughout the test. The tests were carried out using a non-sink, gastric transfer model. 750 ml of 0.1 N HCl was added to the vessels and heated to 37° C. 187.5 mg of the milled powder (equivalent to 37.5 mg itraconazole) was added to the vessel and stirred at 75 RPM for 120 minutes. At this point, the volume and pH of media was increased to 1000 ml and pH 6.8 by the addition of a 250 ml aliquot of 0.20 M Na₃PO₄. The dissolution experiment was allowed to run for a further 180 minutes, at which time the experiment was terminated. Samples were collected throughout the experiment at 60, 120, 135, 150, 180, 240, and 300 minutes and analyzed for the amount of itraconazole dissolved via HPLC. The resulting dissolution profile was compared to literature values for the dissolution of itraconazole from Sporanox® capsules (Dinunzio, et al. Molecular Pharmaceutics, Vol. 5 No. 6, pp. 968-980 (2008)) and is shown in Table 2 and Table 3. Area Under the Dissolution Curve (AUDC) was calculated using the trapezoidal rule for the part of the dissolution experiment which took place in the pH 6.8 media and values are also shown in Table 2 and Table 3.

### In Vivo Pharmacokinetic Study

To examine if the in vitro data would translate to an increase in bioavailability in vivo, it was decided to undertake a pharmacokinetic study in a rat model. For the pharmacokinetic study, Composition 2 was utilized and compared to Sporanox® capsules and OnMel® tablets.

### Preparation of Dose

An itraconazole formulation produced by Kinetisol® (equivalent to Composition 2) was prepared as stated above. Sporanox® pellets were extracted from the capsule shells and ground in a mortar and pestle to produce a powder. OnMel® tablets were ground in a mortar and pestle to produce a powder. In all three cases, the powders were passed through a 60-mesh screen to obtain a particle size less than 250 μm.

The dosage vehicle was hydroxypropyl cellulose (2%) and Tween 80 (0.1%) dissolved in water and titrated to pH 2.0.

On the day of the study, each dose formulation was prepared accordingly (Table 4):

For the Group 1 dose formulation, dose vehicle (2% HPC/0.1% Tween 80 in water, pH 2.0, 50.01 g) was weighed into a glass container and stirred rapidly on a magnetic stir plate. KSD powder (1.5003 g) was gradually added to the stirring vehicle solution. The formulation was mixed on the stir plate for 60 minutes and sonicated for 3 minutes in a 25-30° C. water bath to produce a yellow homogeneous suspension at a target concentration of 6 mg API/mL for oral administration.

For Group 2, dose vehicle (50.01 g) was weighed into a glass container and stirred rapidly on a magnetic stir plate. The Sporanox powder (1.3800 g) was gradually added to the stirring vehicle solution. The formulation was mixed on the stir plate for 51 minutes and sonicated for 3 minutes in a 25-30° C. water bath to produce a white homogeneous suspension at a target concentration of 6 mg API/mL for oral administration.

For Group 3, dose vehicle (50.00 g) was weighed into a glass container and stirred rapidly on a magnetic stir plate. The OnMel® powder (1.3802 g) was gradually added to the stirring vehicle solution. The formulation was mixed on the stir plate for 38 minutes and sonicated for 3 minutes in a 25-30° C. water bath to produce a white homogeneous suspension at a target concentration of 6 mg API/mL for oral administration.

The dose formulations were mixed continuously on a magnetic stir plate until the completion of dosing to ensure homogeneity.

### Study Design and Dosing

Twelve male Sprague-Dawley rats were received from Charles River Laboratories (Kingston, N.Y.). Each animal was equipped with a surgically-implanted jugular vein catheter to facilitate blood collection. Twelve animals were assigned to the study based on catheter patency and acceptable health as determined by a staff veterinarian. The animals were placed into three groups of four animals per group. All animals were fasted overnight prior to dose administration and food was returned following the 4-hour post-dose blood collection. The final study design can be found in Table 4.

Each animal received a single administration of the appropriate prepared test article by oral gavage at a target dose level of 30 mg API/kg and at a dose volume of 5 mL/kg. Dose administration data including pre-dose animal body weights are presented in Table 5.

Blood samples (0.25 mL; sodium heparin anticoagulant) were collected from the jugular vein catheter or by venipuncture of a tail vein if the catheter became impatent. Blood samples were collected from each animal at 2, 3, 3.5, 4, 5, 6, 8, 12, and 24 hours following oral dosing. All whole blood samples were placed on wet ice immediately after collection and were centrifuged at 2-8° C. to isolate plasma. The resulting plasma was transferred to individual polypropylene tubes and immediately placed on dry ice until storage at nominally −20° C. before analysis for itraconazole concentration was performed.

The plasma samples were analyzed for itraconazole concentration using a research grade LC-MS/MS Assay.

Pharmacokinetic parameters were estimated from the plasma concentration-time data using standard noncompartmental methods and utilizing suitable analysis software (Watson 7.2 Bioanalytical LIMS, Thermo Electron Corp).

TABLE 1
Formulations, Processing Parameters, and
Characterization of Compositions 1-11.

| | | | | PXRD | |
|---|---|---|---|---|---|
| | | Processing | Ejection | Characterization | |
| | | Speed | Tem- | (Amorphous/ | |
| Composition | Polymer | (RPM)/ | perature | Crystalline) | |
| (ITZ:PVA:other) | Type | Time (sec) | (° C.) | PVA | Itraconazole |
| 1 (1:4) | 4-88 | 2400- | 185 | X | A |
| | | 3000/12 | | | |
| †2 (1:4) | 4-88 | 2400/13 | 150 | X | A |
| 3 (1:2) | 4-88 | 2400- | 185 | X | A |
| | | 3000/12 | | | |
| 4 (1:4) | 4-38 | 2400/9 | 150 | A | A |
| 5 (1:4) | 4-75 | 2400/9 | 150 | X | A |
| 6 (1:4) | 4-98 | 2400/14 | 120 | X | A |
| 7 (1:4) | 26-88 | Not Processable | | | |
| 8 (1:4) | 40-88 | Not Processable | | | |
| 9 (1:2:2) | 4-88, | 2400- | 185 | X | A |
| | 26-88 | 3000/12 | | | |
| 10 (1:2:2) | 4-88, | 2400- | 185 | X | A |
| | 40-88 | 3000/12 | | | |
| 11 (2:5:3) | 4-88, | 2400/10 | 150 | X | A |
| | PVP K25 | | | | |

†Low Temperature Composition;
X = Crystalline;
A = Amorphous

TABLE 2
Tabulated dissolution data from Sporanox and ITZ Kinetisol ® compositions (1-5).

| Time | | | | | | |
|---|---|---|---|---|---|---|
| (min) | Sporanox ® | Comp. 1 | Comp. 2 | Comp. 3 | Comp. 4 | Comp. 5 |
| 0 | | 0 | 0 | 0 | 0 | 0 |
| 60 | | 33.78 ± 1.26 | 34.64 ± 0.38 | 34.15 ± 0.80 | 12.82 ± 1.67 | 35.33 ± 0.45 |
| 120 | | 35.52 ± 1.32 | 36.22 ± 0.52 | 37.12 ± 0.50 | 15.21 ± 1.16 | 37.06 ± 0.27 |
| (0)** | | | | | | |
| 135 | | 7.77 ± 5.60 | 8.67 ± 3.17 | 6.50 ± 2.11 | 0.01 ± 0.02 | 5.41 ± 0.68 |
| (15)** | | | | | | |
| 150 | | 4.22 ± 3.71 | 4.75 ± 1.09 | 2.50 ± 0.62 | 0.006 ± 0.02 | 1.62 ± 0.84 |
| (30)** | | | | | | |
| 180 | | 2.22 ± 1.17 | 1.44 ± 1.08 | 0.65 ± 0.10 | 0.012 ± 0.013 | 0.60 ± 0.27 |
| (60)** | | | | | | |
| 240 | | 0.74 ± 0.45 | 0.88 ± 0.3 | 0.26 ± 0.10 | 0 | 0.27 ± 0.07 |
| (120)** | | | | | | |
| 300 | | 0.14 ± 0.12 | 0.45 ± 0.14 | 0.12 ± 0.04 | 0 | 0.03 ± 0.02 |
| (180)** | | | | | | |
| AUC | 225.8 ± 23.1* | 626 ± 202 | 640 ± 89 | 480 ± 31 | 115 ± 8 | 442 ± 23 |
| (mg · min) | | | | | | |

All dissolution data reported as mg of drug in solution
*from Dinunzio, et al. Molecular Pharmaceutics, Vol. 5 No. 6, pp. 968-980 (2008)
**Time in minutes used to calculate area under dissolution curve in pH 6.8 phosphate buffer

TABLE 3
Tabulated dissolution data from Sporanox and ITZ
Kinetisol ® compositions (6-11).

| Time | | | | | |
|---|---|---|---|---|---|
| (min) | Sporanox ® | Comp. 6 | Comp. 9 | Comp. 10 | Comp. 11 |
| 0 | | 0 | 0 | 0 | 0 |
| 60 | | 9.98 ± | 35.33 ± | 38.68 ± | 32.76 ± |
| | | 0.51 | 0.45 | 0.59 | 1.19 |
| 120 | | 17.09 ± | 37.06 ± | 41.96 ± | 35.48 ± |
| (0)** | | 1.00 | 0.27 | 0.997 | 0.26 |
| 135 | | 0.01 ± | 5.41 ± | 3.83 ± | 3.41 ± |
| (15)** | | 0.001 | 0.68 | 2.13 | 0.92 |
| 150 | | 0.01 ± | 1.62 ± | 1.02 ± | 1.89 ± |
| (30)** | | 0.01 | 0.84 | 0.82 | 0.20 |
| 180 | | 0.003 ± | 0.60 ± | 0.50 ± | 0.58 ± |
| (60)** | | 0.001 | 0.27 | 0.35 | 0.04 |
| 240 | | 0.01 ± | 0.27 ± | 0.66 ± | 0.17 ± |
| (120)** | | 0.02 | 0.07 | 0.90 | 0.02 |
| 300 | | 0.02 ± | 0.12 ± | 1.40 ± | 0.19 ± |
| (180)** | | 0.01 | 0.03 | 1.22 | 0.07 |
| AUC | 225.8 ± | 130 ± | 442 ± | 115 ± | 400 ± |
| (mg · min) | 23.1* | 9 | 23 | 8 | 17 |

All dissolution data reported as mg of drug in solution
*from Dinunzio, et al. Molecular Pharmaceutics, Vol. 5 No. 6, pp. 968-980 (2008)
**Time in minutes used to calculate area under dissolution curve in pH 6.8 phosphate buffer

TABLE 4
Final Pharmacokinetic Study Design

| | | Treatment | | | | | |
|---|---|---|---|---|---|---|---|
| | | | | | Dose | | |
| Group | No. of | Test | Dose Level | Dose Conc. | Volume | Dose | Dose |
| No. | Males | Article | (mg API/kg) | (mg API/kg) | (mL/kg) | Vehicle | Route |
| 1 | 4 | KSD | 30 | 6 | 5 | 2% HPC/ | oral |
| | | [ITZ:PVA 4-88 | | | | 0.1% | gavage |
| | | (1:4)] | | | | Tween | |
| 2 | 4 | Sporanox | 30 | 6 | 5 | 80/ | |
| | | pellets | | | | pH 2.0 | |
| | | (pulverized) | | | | | |
| 3 | 4 | OnMel pellets | 30 | 6 | 5 | | |
| | | (pulverized) | | | | | |

HPC = Hydroxypropyl cellulose

TABLE 5
Dose Administration

| | | | | | Animal | Formulation | Dose | Dose | Dose | Target |
|---|---|---|---|---|---|---|---|---|---|---|
| Test | Group | Study | | Dose | Weight | Administered | Conc.^{a} | Administered | Administered | Dose |
| Article | ID | ID | Sex | Route | (kg) | (mL) | (mg/mL) | (mg) | (mg/kg) | (mg/kg) |
| KSD | 1 | 1 | M | Oral | 0.257 | 1.29 | 6 | 7.74 | 30.12 | 30 |
| | | | | Gavage | | | | | | |
| KSD | 1 | 2 | M | Oral | 0.251 | 1.26 | 6 | 7.56 | 30.12 | 30 |
| | | | | Gavage | | | | | | |
| KSD | 1 | 3 | M | Oral | 0.243 | 1.22 | 6 | 7.32 | 30.12 | 30 |
| | | | | Gavage | | | | | | |
| KSD | 1 | 4 | M | Oral | 0.254 | 1.27 | 6 | 7.62 | 30 | 30 |
| | | | | Gavage | | | | | | |
| Sporanox | 2 | 5 | M | Oral | 0.246 | 1.23 | 6 | 7.38 | 30 | 30 |
| | | | | Gavage | | | | | | |
| Sporanox | 2 | 6 | M | Oral | 0.246 | 1.23 | 6 | 7.38 | 30 | 30 |
| | | | | Gavage | | | | | | |
| Sporanox | 2 | 7 | M | Oral | 0.244 | 1.22 | 6 | 7.32 | 30 | 30 |
| | | | | Gavage | | | | | | |
| Sporanox | 2 | 8 | M | Oral | 0.259 | 1.3 | 6 | 7.8 | 30.12 | 30 |
| | | | | Gavage | | | | | | |
| Onmel | 3 | 12 | M | Oral | 0.235 | 1.18 | 6 | 7.08 | 30.13 | 30 |
| | | | | Gavage | | | | | | |

TABLE 6
Pharmacokinetic Summary

| | | | | | | | | | Bioavailability |
|---|---|---|---|---|---|---|---|---|---|
| | | Dose | AUC_{0-24 h} | AUC_{0-inf} | Cₘₐₓ | Tₘₐₓ | T_{1/2} | | Compared |
| | n | (mg/kg) | (h · ng/ml) | (h · ng/ml) | (ng) | (ng) | (h) | % RSD | to Sporanox |
| ITZ:PVA | 3 | 30.12 | 2943.32 ± 1359.75 | 2990.00 ± 1396.32 | 417.33 ± 119.26 | 3.83 ± 0.29 | 3.55 ± 0.64 | 46.7 | 3.90 |
| (1:4) | | | | | | | | | |
| Sporanox | 4 | 30.03 | 746.25 ± 410.53 | 765.75 ± 398.95 | 127.30 ± 48.66 | 3.50 ± 0.41 | 3.00 ± 0.23 | 52.1 | 1.00 |
| OnMel | 4 | 30.03 | 2564.75 ± 1373.91 | 2624.25 ± 1390.75 | 366.50 ± 201.81 | 3.50 ± 1.22 | 4.18 ± 0.89 | 53.0 | 3.42 |

### Results and Discussion

### Processing

Compositions 1-6 all processed with no adverse events, utilizing material ejection temperatures in the range of 120-180° C. From the processing times, it can be seen that Compositions 1-3 and 6, comprising PVA 4-88 and PVA 4-98, respectively, had longer processing times than Compositions 4 and 5, comprising PVA 4-38 and 4-75, respectively. The increase in processing time is likely due to the higher degree of crystallinity in the 88% and 98% hydrolyzed grades. More energy and shear forces are needed to disrupt the crystallinity of the polymer, which translates to an increase in manufacturing time.

It was not possible to create transparent/translucent blends with Compositions 7 and 8, PVA 26-88 and 40-88, respectively. The masses, which were ejected, were of dark brown in color, indicating some sort of damage occurred during processing to either the polymer, the ITZ, or both. A likely reason for the discoloration is that the increase in polymer chain length led to a higher degree of polymer chain entanglement during the processing, resulting in an increase in heat followed by thermal degradation. A contributing factor to this could also be the presence of a higher amount of crystallinity in the long chain polymers compared to the short chain polymers.

To overcome this issue, the long chain polymers were blended with the short chain PVA 4-88 in a 1:1 ratio. The addition of the short chain polymer overcame the issue of discoloration and transparent/translucent compositions were achievable (Compositions 9 and 10).

Finally, PVA 4-88 was blended with PVP K25 (Composition 11) to investigate whether the addition of the PVP K25 would lead to higher supersaturated states of ITZ in neutral media.

### Characterization

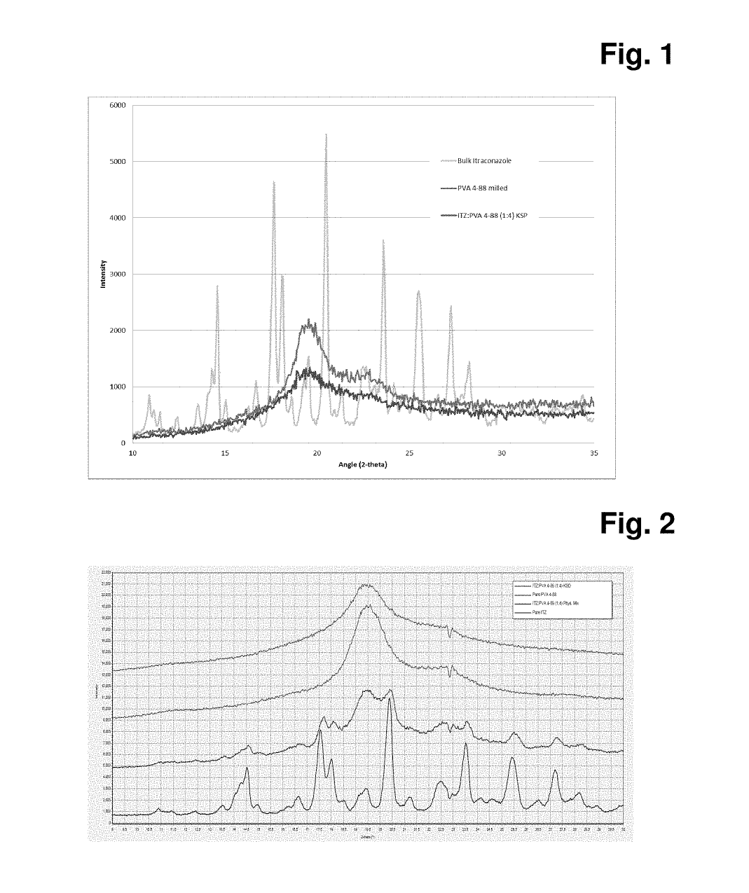

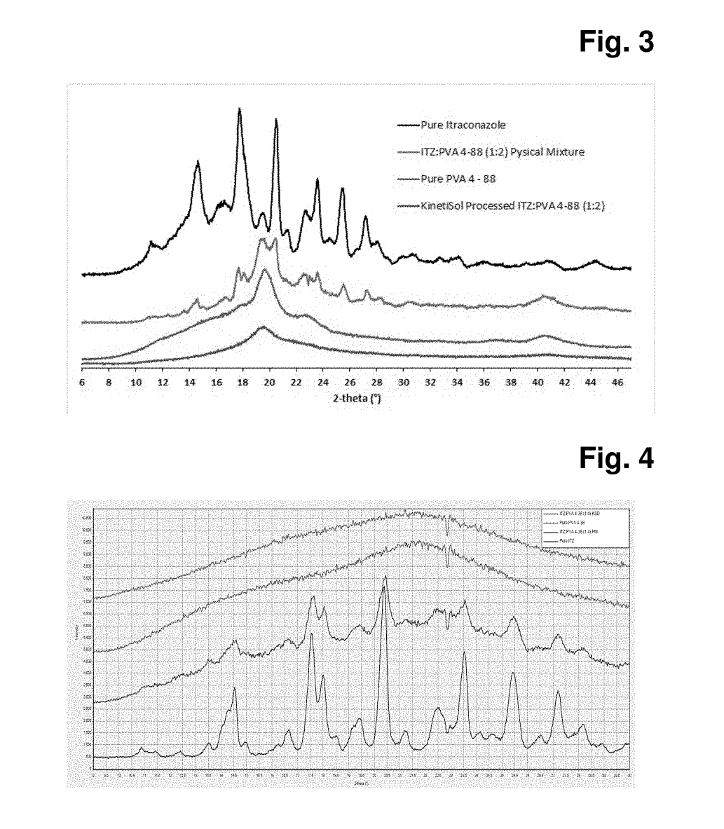

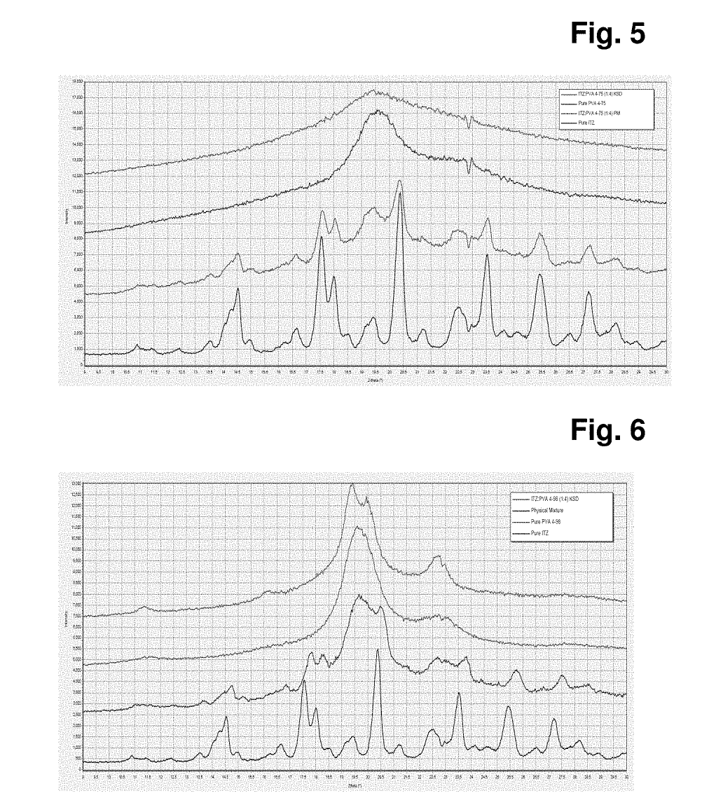

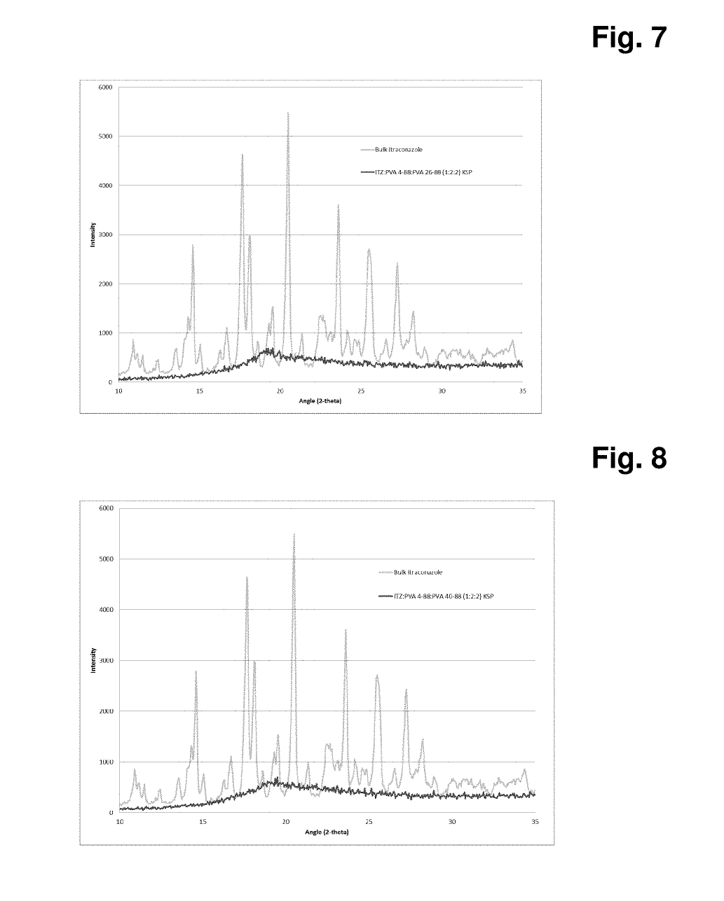

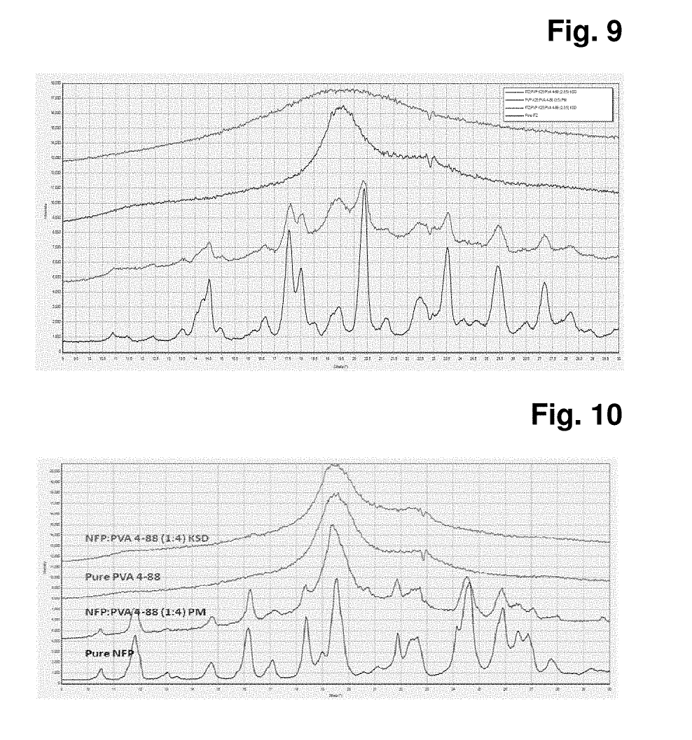

For all Compositions investigated (FIG. 1, FIG. 2, FIG. 3, FIG. 5, FIG. 6, FIG. 7, FIG. 8, FIG. 8, FIG. 9), outside of Composition 4 (FIG. 4), the XRD diffractograms showed a broad diffraction peak in the area of 19.5° (2-theta) indicating the PVA in existed in a semi-crystalline state. No peaks corresponding peaks were found for ITZ, indicating the API was present in an amorphous state. For Composition 4, which utilized PVA 4-38, both the polymer and ITZ were amorphous. The high amount of acetate groups present in the polymer are responsible for the amorphous nature. As the number of acetate functional groups decreases, the crystallinity of the polymer increases and it can be seen that the maxima at 19.5° (2-theta) begins to narrow and increase in height as the percent hydroxylation of the PVA increases.

FIG. 1: XRD diffractogram overlay of Composition 1 KinetiSol® (KSD) product (180° C. ejection temperature) with pure PVA 4-88, and pure ITZ.

FIG. 2: XRD diffractogram overlay of Composition 2 KinetiSol® (KSD) product (150° C. ejection temperature) with the corresponding physical mixture, pure PVA 4-88, and pure ITZ.

FIG. 3: XRD diffractogram overlay of Composition 2 KinetiSol® (KSD) product with the corresponding physical mixture, pure PVA 4-98, and pure ITZ

FIG. 4: XRD diffractogram overlay of Composition 4 KinetiSol® (KSD) product with the corresponding physical mixture, pure PVA 4-38, and pure ITZ.Composition.

FIG. 5: XRD diffractogram overlay of Composition 5 KinetiSol® (KSD) product with the corresponding physical mixture, pure PVA 4-75, and pure ITZ.Composition.

FIG. 6: XRD diffractogram overlay of Composition 6 KinetiSol® (KSD) product with the corresponding physical mixture, pure PVA 4-98, and pure ITZ.

FIG. 7: XRD diffractogram overlay of Composition 9 KinetiSol® (KSD) product with pure ITZ.Composition.Composition 9.

FIG. 8: XRD diffractogram overlay of Composition 10 KinetiSol® (KSD) product with pure ITZ.Composition.

FIG. 9: XRD diffractogram overlay of Composition 11 KinetiSol® (KSD) product with the corresponding physical mixture, PVA 4-88/PVP K25 mixture, and pure ITZ

### Dissolution

A comparative dissolution analysis of the four KinetiSol® processed ITZ-PVA compositions can be revisited in Table 2 and Table 3. The dissolution results of Sporanox pellets, as reported by Dinunzio (Dinunzio, et al. Molecular Pharmaceutics, Vol. 5 No. 6, pp. 968-980 (2008)), show that 37.5 mg of ITZ are dissolved in the acidic phase of the test at the 120 minute timepoint, which equals 100% drug release. When the media is neutralized to pH 6.8, there is a decrease in fraction ITZ dissolved to about 2 mg at the 15 minute timepoint after the pH changed, followed by essentially no itraconazole dissolved at the 30 minute timepoint after the pH change. This is due to the weakly basic drug being more soluble in acidic media than neutral media. In the neutral media, where the drug is less soluble, the decreasing fraction dissolved can be explained by nucleation and recrystallization of the drug due to an unstable supersaturated state.

Although all of the PVA formulations exhibit a decrease in fraction of drug dissolved in the neutral phase of the dissolution study, this decrease in rate is much slower than the Sporanox formulation in the case of Compositions 1, 2, 3, 5, 9, and 11, due to the concentration enhancing properties of PVA (also, 100% (37.5 mg ITZ) of the theoretical drug load was recovered at the end of the acid testing for these formulations). In fact, Composition 2, which had an ejection temperature of 150° C. (lower than the melting point of both polymer and drug) exhibited a much narrower standard deviation of the Area Under the Dissolution Curve (AUDC) in the neutral media than Composition 1, which had an ejection temperature of 180° C. Composition 3, which was a 1:2 mixture of ITA:PVA, also had a very high AUDC in neutral media, indicating that increasing the drug concentration in the formulation does not have a significant negative effect in neutral media. Composition 4 achieved only about 15 mg of drug dissolved in the acid phase, followed by very poor performance in the neutral media. The poor performance in the acid phase was due to poor wettability of powder and the insoluble characteristic of the PVA 4-38, which consists of 38% hydroxyl functional groups and 62% hydrophobic acetate groups. Composition 6, as well, exhibited poor dissolution in the acidic phase, followed by poor performance in the neutral phase; however, this was not due to the poor wettability of the PVA 4-98, but the slow dissolution kinetics of the polymer.

It is well known that as the number of hydroxyl groups increases, the crystallinity of the polymer increases as well. However, this increase in crystallinity and hydrophilicity of the polymer is accompanied by a decrease in dissolution kinetics of the highly crystalline polymer. Composition 10, although reaching, theoretically, 100% of ITZ dissolved in the acidic phase of the dissolution study, exhibited a very fast decrease in the fraction of ITZ dissolved in the neutral phase of the dissolution study. In this case, the higher molecular weight PVA 40-88 did not possess the same concentration enhancing effects the lower molecular weight PVA's exhibited.

Comparing the AUDC of Compositions 1-11 to that of Sporanox capsules, reported by Dinunzio (Dinunzio, et al. Molecular Pharmaceutics, Vol. 5 No. 6, pp. 968-980 (2008)), Composition 2 had an AUDC 2.45 times higher than Sporanox and was selected for further in vivo studies.

### In Vivo Pharmacokinetic Study

Pharmacokinetic parameters appropriate for the available plasma data and dose route (AUC₀₋₂₄, AUC_{0-inf}, Cₘₐₓ, Tₘₐₓ, T_{1/2}) can be found in Table 6. Sporanox capsules contain 100 mg of ITZ per dose and is intended to be taken twice daily.

The formulation is manufactured by dissolving hypromellose and ITZ in a common solvent or co-solvent system. The solubilized drug and polymer are then layered onto inactive pellets, creating a solid dispersion of ITZ in hypromellose. OnMel® once-daily tablets contain 200 mg of ITZ per dose, have an equivalent bioavailability as 100 mg twice-daily Sporanox capsules, and are produced via a melt extrusion method using hypromellose as the continuous phase and propylene glycol as a plasticizer, as well as other excipients which are used to formulate the tablet dosage form.

As mentioned previously, Composition 2 was selected as the model to proceed in the pharmacokinetic study, as the AUDC was 2.45 times higher in the neutral phase of the dissolution experiment than Sporanox. It was believed that a higher AUDC in the neutral phase of the dissolution study should relate to a higher bioavailability in vivo.

As can be seen in Table 6, Composition 2, the Area Under the Curve of the plasma vs. time data from zero to infinity (AUC_{0-inf}) was 3.9 times greater than that of the Sporanox capsules and somewhat higher, although not significantly higher, than that of OnMel® tablets and should be considered equivalent. Although the amount of variation in Composition 2 seems to be quite high, we can also see from Table 6 that is of the same order as the two commercially produced dosage forms.

### Nifedipine (NIF) Compositions

### Methods

### Processing

Nifedipine, PVA, and PVP K25 (when used) (Table 7) were first blended in a powder mixer to achieve blend uniformity. The resulting mixture was then dosed into the Kinetisol® compounder and processed according to the conditions found in Table 7. Upon reaching the pre-set ejection temperature, the material was ejected from the compounder, pressed into a disc, and allowed to cool to room temperature. Following quenching, composition was milled using a Fitzpatrick L1A FitzMill. The mill was operated in hammer forward orientation, fitted with a 500 μm screen at an operating RPM of 5,000. The particles which passed through a 250 μm sieve were selected for further testing.

### Characterization

NIF-PVA KinetiSol processed samples and the corresponding controls were analyzed for crystalline character by x-ray diffraction (XRD) using an Equinox 100 benchtop x-ray diffractometer (Inel, Inc., Stratham, N.H.). Samples were placed in an aluminum crucible and loaded into a rotating sample holder. Samples were analyzed for 600 seconds using a Cu K radiation source (λ=1.5418 Å) operating at 42 kV and 0.81 mA in a 2-theta range of 0-150°. Results are reported from 5-30° (2-theta) as this is the primary region for the diffraction of x-rays by NIF.

### Dissolution

All dissolution tests were conducted in a Vankel VK-7000 (Varian, Inc.) USP apparatus II (paddle) dissolution tester. The dissolution medium was maintained at 37° C. with a recirculating heater and a paddle speed of 50 RPM was constant throughout the test. The theoretical equivalent of 90 mg of NIF (450 mg of KSD product), an amount equal to 10× the thermodynamic solubility, was added to each vessel containing 900 ml of the dissolution medium (n=3). The dissolution medium utilized was USP pH 6.8 phosphate buffered solution. The dissolution medium was degassed prior to testing by the heat/sonication method.

The concentration of NIF in solution during the 180 minute dissolution test is shown in Table 8. Samples were collected throughout the experiment at 15, 30, 60, 120, and 180 minutes. The samples were filtered through 200 nm PTFE syringe filters, diluted 1:1 with acetonitrile and analyzed for the amount of nifedipine dissolved via HPLC. The resulting dissolution profiles were compared to literature values for the dissolution of nifedipine from a previously published report (Tanno, et al. Drug Development and Industrial Pharmacy, Vol. 30 No. 1, pp. 9-17 (2004)).

TABLE 7
Formulations, Processing Parameters, and
Characterization of Compositions 12-15

| | | | | PXRD | |
|---|---|---|---|---|---|
| | | Processing | Ejection | Characterization | |
| | | Speed | Tem- | (Amorphous/ | |
| Composition | Polymer | (RPM)/ | perature | Crystalline) | |
| (NIF:PVA:other) | Type | Time (sec) | (° C.) | PVA | Nifedipine |
| 12 (1:4) | 4-88 | 2000/11 | 110 | X | A |
| 13 (1:4) | 4-38 | 2000/10 | 110 | A | A |
| 14 (1:4) | 4-75 | 2000/9 | 110 | X | A |
| 15 (2:5:3) | 4-88, | 2000/10 | 130 | X | A |
| | PVP K25 | | | | |

X = Crystalline;
A = Amorphous

TABLE 8
Tabulated dissolution data from NIF Kinetisol compositions.

| Time | | | | | |
|---|---|---|---|---|---|
| (min) | Nifedipine | Comp. 12 | Comp. 13 | Comp. 14 | Comp. 15 |
| 0 | Equilibrium | 0 | 0 | 0 | 0 |
| 15 | Solubility | 13.36 ± | 1.81 ± | 4.6 ± | 15.83 ± |
| | ca. 9 μg/ml* | 0.08 | 0.47 | 0.83 | 0.49 |
| 30 | | 15.23 ± | 2.35 ± | 8.98 ± | 15.73 ± |
| | | 0.30 | 0.53 | 2.14 | 0.44 |
| 60 | | 14.17 ± | 2.93 ± | 11.98 ± | 15.24 ± |
| | | 1.23 | 0.55 | 1.92 | 0.36 |
| 120 | | 13.5 ± | 4.19 ± | 14.14 ± | 14.42 ± |
| | | 1.16 | 0.98 | 1.28 | 0.25 |
| 180 | | 14.11 ± | 5.14 ± | 12.98 ± | 14.00 ± |
| | | 0.39 | 1.07 | 1.94 | 0.41 |

All dissolution data reported as μg/ml
*from Tanno, et al. Drug Development and Industrial Pharmacy, Vol. 30 No. 1, pp. 9-17 (2004)

### Results and Discussion

### Processing

An ejection temperature of 110° C. was found to be optimal for compositions 12-14. Due to the addition of PVP K25 in Composition 15, the ejection temperature needed to be increased to 130° C. to render NIF amorphous.

### Characterization

The results of XRD analysis of Composition 12 KinetiSol® product are shown in FIG. 10. It is seen in this figure that the KinetiSol® product is XRD amorphous with respect to NIF. It is also seen in this analysis that pure PVA 4-88 is moderately crystalline, described by a broad diffraction peak with a maxima at 19.5° 2-theta.

FIG. 10: XRD diffractogram overlay of Composition 12 KinetiSol® (KSD) product with the corresponding physical mixture (PM), pure PVA 4-88, and pure NIF

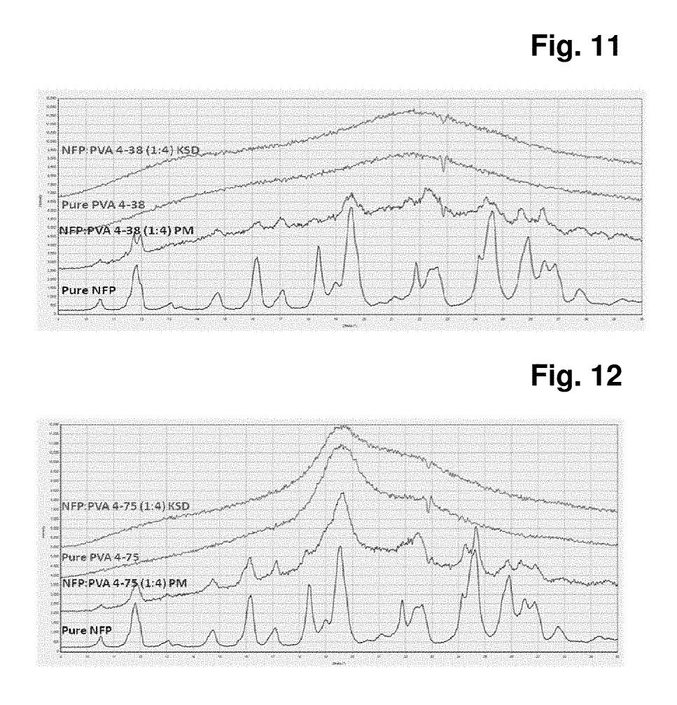

The results of XRD analysis of Composition 13 KinetiSol® product are shown in FIG. 11. It is seen in this figure that the KinetiSol® product is entirely amorphous exhibiting none of the characteristic crystalline peaks of NIF. It is also seen that pure PVA 4-38 is entirely XRD amorphous.

FIG. 11: XRD diffractogram overlay of Composition 13 KinetiSol® (KSD) product with the corresponding physical mixture (PM), pure PVA 4-38, and pure NIF

The results of XRD analysis of Composition 14 KinetiSol® product are shown in FIG. 12. It is seen in this figure that the KinetiSol®l product is XRD amorphous with respect to NIF. It is also seen in this analysis that pure PVA 4-75 is moderately crystalline, described as a broad diffraction peak with a maxima at about 19.5° 2-theta.

FIG. 12: XRD diffractogram overlay of Composition 14 KinetiSol® (KSD) product with the corresponding physical mixture (PM), pure PVA 4-75, and pure NIF

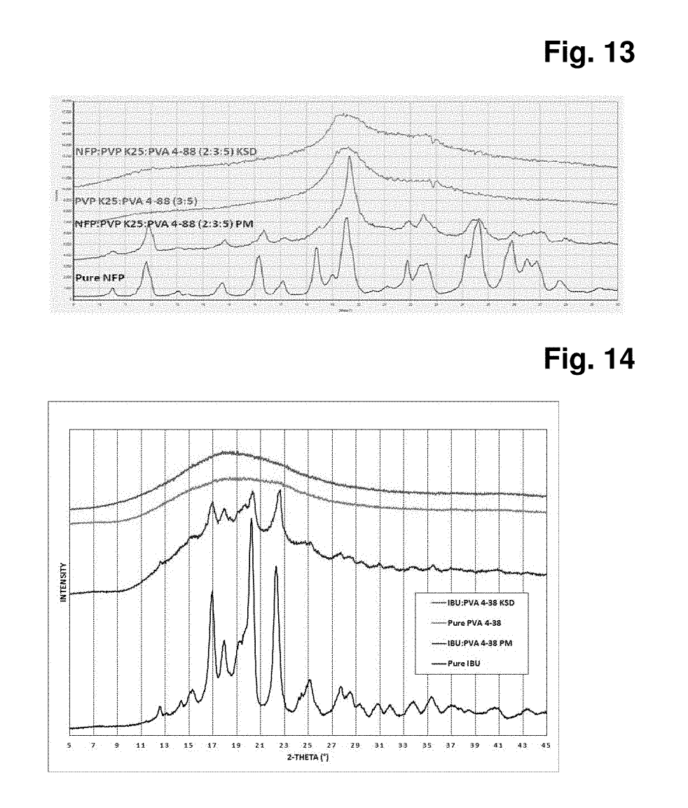

The results of XRD analysis of Composition 15 KinetiSol® product are shown in FIG. 13. It is seen in this figure that the KinetiSol® product is XRD amorphous with respect to NIF and PVP K25. It is also seen in this analysis that pure PVA 4-88 is moderately crystalline, described as a broad diffraction peak with a maxima at about 19.5° 2-theta.

FIG. 13: XRD diffractogram overlay of Composition 15 KinetiSol® (KSD) product with the corresponding physical mixture (PM), pure PVA 4-88/PVP K25 KinetiSol® product, and pure NIF

### Dissolution

A comparative dissolution analysis of the four KinetiSol® processed NIF-PVA products can be revisited in Table 8. From previously published literature (Tanno, et al. Drug Development and Industrial Pharmacy, Vol. 30 No. 1, pp. 9-17 (2004)), the maximum solubility of NIF in pH 6.8 buffer is about 9 μg/ml. Composition 12 wetted and dispersed well and achieved a 1.52-fold supersaturation at Cₘₐₓ (15.23 μg/ml). The Tₘₐₓ was 30 minutes, followed by a drop in dissolved drug to about 14 μg/ml at the 180 minute timepoint. Composition 13 wetted poorly and formed a gellated mass upon introduction to the dissolution vessel. This is most likely due to the insolubility of the 38% hydrolyzed grade of PVA used for the composition. The NIF was released slowly and the Cₘₐₓ was 51% (5.14 μg/ml) of the thermodynamic solubility of NIF and Tₘₐₓ was at the 180 minute timepoint. Composition 14 wetted and dispersed slowly. This is reflected by the slow achievement of the Cₘₐₓ (14.14 μg/ml) and Tₘₐₓ (120 minutes). This is probably due to the fact that polyvinyl alcohols with lower grades of hydrolysis are generally not as soluble as those with a higher grade of hydrolysis. The formulation achieved a 1.41-fold supersaturation at Cₘₐₓ. Finally, Composition 15 wetted and dispersed well. This is due to the addition of the water soluble polymer polyvinyl pyrrolidone. At Cₘₐₓ (15.83 μg/ml), there was a 1.58-fold supersaturation and the Tₘₐₓ (15 minutes) was reached faster than all other compositions studied. The fast and high degree of supersaturation is attributed to the addition of the very soluble PVP K25 added to the formulation.

### Ibuprofen (IBU) Compositions

### Methods

### Processing

Ibuprofen, PVA, and PVPVA 64 (when used) Table 9) were first blended in a powder mixer to achieve blend uniformity. The resulting mixture was then dosed into the Kinetisol® compounder and processed according to the conditions found in Table 9. Upon reaching the pre-set ejection temperature, the material was ejected from the compounder, pressed into a disc, and allowed to cool to room temperature. Following quenching, composition was milled using a Fitzpatrick L1A FitzMill. The mill was operated in hammer forward orientation, fitted with a 500 μm screen at an operating RPM of 5,000. The particles which passed through a 250 μm sieve were selected for further testing.

### Characterization

IBU-PVA KinetiSol® processed samples and the corresponding controls were analyzed for crystalline character by x-ray diffraction (XRD) using an Equinox 100 benchtop x-ray diffractometer (Inel, Inc., Stratham, N.H.). Samples were placed in an aluminum crucible and loaded into a rotating sample holder. Samples were analyzed for 600 seconds using a Cu K radiation source (λ=1.5418 Å) operating at 42 kV and 0.81 mA in a 2-theta range of 0-150°. Results are reported from 5-45° (2-theta) as this is the primary region for the diffraction of x-rays by IBU.

### Dissolution

All dissolution tests were conducted in a Vankel VK-7000 (Varian, Inc.) USP apparatus II (paddle) dissolution tester. The dissolution medium was maintained at 37° C. with a recirculating heater and a paddle speed of 75 RPM was constant throughout the test. The theoretical equivalent of 200 mg of IBU (1.0 g of KSD product) was added to each vessel containing 1 L of the dissolution medium (n=3). The KSD product was passed through an 850 μm screen for delumping prior to dispensing. The dissolution medium recipe (SGFsp) consisted of 2.0 g NaCl and 80 ml of 0.1 N HCl in a total of 1 L of purified water (pH=1.2). The dissolution medium was degassed prior to testing by the heat/sonication method.

The concentration of IBU in solution during the 120 minute dissolution test (shown in Table 10) was measured once per minute using a Pion Spectra in-situ fiber-optic UV-diss system (Pion, Inc., Billerica, Mass.) with a 1 mm path length probe tip. Concentrations were determined by integrating the area under the UV absorption curve in a wavelength range of 216 to 222 nm with a baseline correction at 450 nm. Linearity was established over a range of 1.4 to 200 μg/ml with a correlation coefficient of 0.9992. A diluents consisting of 7:3 (V/V) SGFsp:Acetonitrile (HPLC grade, no UV absorption beyond 190 nm) was used to generate the IBU standard curve.

TABLE 9
Formulations, Processing Parameters, and
Characterization of Compositions 16-22.

| | | | Ejec- | PXRD | |
|---|---|---|---|---|---|
| Compo- | | | tion | Characterization | |
| sition | | Processing | Temper- | (Amorphous/ | |
| (IBU:PVA:PV | Polymer | Speed (RPM)/ | ature | Crystalline) | |
| PVA 64) | Type | Time (sec) | (° C.) | PVA | Nifedipine |
| 16 (1:4) | 4-38 | 3000/8 | 110 | A | A |
| 17 (1:4) | 4-75 | 3000/— | 110 | Purity too low | |
| 18 (1:4) | 4-88 | 1500- | 110-140 | Purity too low | |
| | | 3600/— | | | |
| 19 (1:4) | 4-98 | 2800- | 110-170 | Purity too low | |
| | | 3600/— | | | |
| 20 (2:7:1) | 4-75, | 2400/10 | 80 | X | A |
| | PVPVA | | | | |
| | 64 | | | | |
| 21 (2:7:1) | 4-88, | 2400/12 | 80 | X | A |
| | PVPVA | | | | |
| | 64 | | | | |
| 22 (2:7:1) | 4-88, | 2400/12 | 80 | X | A |
| | PVPVA | | | | |
| | 64 | | | | |

X = Crystalline;
A = Amorphous

TABLE 10
Tabulated dissolution data from IBU KinetiSol ® compositions

| Time | Pure | | | | |
|---|---|---|---|---|---|
| (min) | Ibuprofen | Comp. 16 | Comp. 20 | Comp. 21 | Comp. 22 |
| 5 | 0.66 ± | 8.68 ± | 46.16 ± | 55.00 ± | 33.61 ± |
| | 0.48 | 4.30 | 13.07 | 1.60 | 1.06 |
| 10 | 2.64 ± | 13.07 ± | 69.12 ± | 69.42 ± | 47.51 ± |
| | 0.43 | 3.13 | 7.55 | 2.12 | 0.89 |
| 15 | 3.38 ± | 16.89 ± | 78.78 ± | 74.19 ± | 54.18 ± |
| | 0.13 | 3.43 | 6.70 | 1.69 | 2.32 |
| 20 | 4.40 ± | 19.55 ± | 84.67 ± | 77.11 ± | 58.63 ± |
| | 0.33 | 3.46 | 6.72 | 2.47 | 0.50 |
| 25 | 6.40 ± | 21.67 ± | 88.68 ± | 77.06 ± | 61.62 ± |
| | 0.67 | 2.97 | 6.78 | 1.18 | 1.19 |
| 30 | 6.42 ± | 24.75 ± | 90.54 ± | 77.41 ± | 64.69 ± |
| | 1.18 | 2.26 | 6.18 | 1.56 | 0.55 |
| 45 | 10.63 ± | 29.50 ± | 92.76 ± | 77.84 ± | 71.07 ± |
| | 1.84 | 2.87 | 5.78 | 1.33 | 0.81 |
| 60 | 14.19 ± | 34.43 ± | 91.92 ± | 78.24 ± | 73.67 ± |
| | 1.46 | 2.55 | 6.48 | 1.38 | 0.31 |
| 75 | 17.63 ± | 37.09 ± | 90.28 ± | 77.69 ± | 75.24 ± |
| | 1.65 | 2.94 | 6.62 | 1.01 | 0.94 |
| 90 | 19.00 ± | 40.55 ± | 88.65 ± | 77.95 ± | 77.23 ± |
| | 1.02 | 3.40 | 6.75 | 0.58 | 1.73 |
| 105 | 21.85 ± | 41.91 ± | 88.84 ± | 77.07 ± | 77.92 ± |
| | 1.40 | 2.77 | 7.78 | 0.84 | 2.19 |
| 120 | 23.98 ± | 43.29 ± | 87.67 ± | 77.23 ± | 78.36 ± |
| | 0.90 | 3.24 | 7.39 | 1.47 | 3.17 |

All dissolution data reported as μg/ml

### Results and Discussion

### Processing

It was found during processing of the IBU-PVA binary systems, that the ability to produce homogenous amorphous product (with respect to IBU) was largely dependent on the degree of hydrolysis (as it relates to percent crystallinity) of the PVA grade. The predominantly amorphous PVA 4-38 grade was found to process well with IBU, yielding an amorphous composition with acceptable purity in a single iteration. The same was also found when processing IBU with PVA 4-75. However, processing the more crystalline 4-88 and 4-98 grades with IBU was found to be a significant challenge. Despite numerous attempts a varying RPM and ejection temperatures, a homogenous amorphous product with acceptable purity could not be achieved with the IBU compositions containing PVA 4-88 and 4-98.

The processing issues were attributed to the differences in melting points between IBU (77° C.) and the polymers; PVA 4-88 (˜190° C.) and PVA 4-98 (˜220° C.). In order to yield a homogenous amorphous drug-polymer composite, the polymer must be rendered molten before, or near, the melting point of the drug. Because PVA 4-38 and 4-75 are largely amorphous, these polymers were able to be softened by the process near the melt transition of IBU. Therefore, the polymers were able to absorb IBU in the molten state to yield an amorphous composition. Alternatively, the PVA 4-88 and 4-98 grades are largely crystalline and were not able to be softened by the process near the melt transition of IBU. Consequently, once melted, IBU acted as a lubricant that prevented generation of the necessary shear and frictional energy needed to render the polymer molten within a processing time and/or at temperature that would not degrade IBU.

To overcome this issue, PVPVA 64 was added to the IBU compositions with PVA 4-88 and 4-98 at a level of 10% (w/w) to provide a plasticizing and binding effect that allowed for the generation of homogenous amorphous product at ejection temperatures as low as 80° C. Although, the binary IBU:PVA 4-75 KinetiSol® composition was found to be acceptable, PVPVA 64 was also included to this formulation so as to not convolute the dissolution comparisons between the key PVA grades.

### Characterization

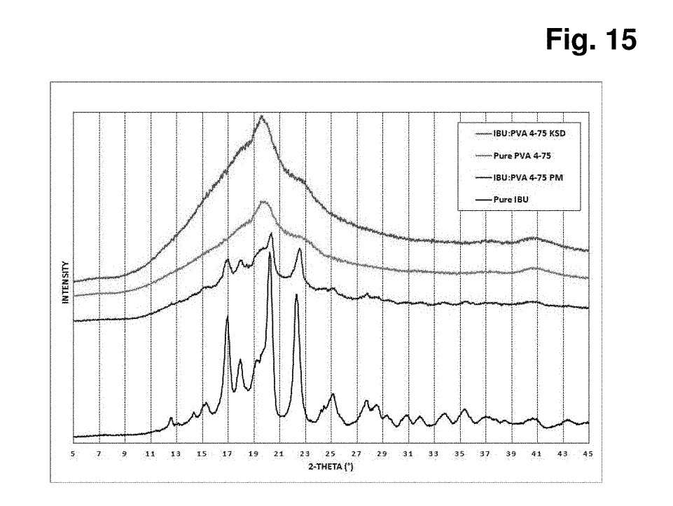

The results of XRD analysis of Composition 16 KinetiSol® product are shown in FIG. 14. It is seen in this figure that the KinetiSol® product is entirely amorphous exhibiting none of the characteristic crystalline peaks of IBU. It is also seen that pure PVA 4-38 is entirely XRD amorphous which substantiates the previous discussion regarding the excellent processability of PVA 4-38 with IBU relative to the more crystalline PVA grades owing to the reduction in processing energy required to render the polymer molten.

FIG. 14: XRD diffractogram overlay of Composition 16 KinetiSol® (KSD) product with the corresponding physical mixture (PM), pure PVA 4-38, and pure IBU.

The results of XRD analysis of Composition 20 KinetiSol® product are shown in FIG. 15. It is seen in this figure that the KinetiSol® product is XRD amorphous with respect to IBU and contains some crystalline character related to the polymer. It is also seen that pure PVA 4-75 is only slightly crystalline which substantiates the previous discussion regarding the good processability of PVA 4-75 with IBU relative to the more crystalline PVA grades owing to the reduction in processing energy required to render the polymer molten.

FIG. 15: XRD diffractogram overlay of Composition 20 KinetiSol® (KSD) product (shown as IBU:PVA 4-75 KSD in the legend for brevity) with the corresponding physical mixture (PM), pure PVA 4-75, and pure IBU.

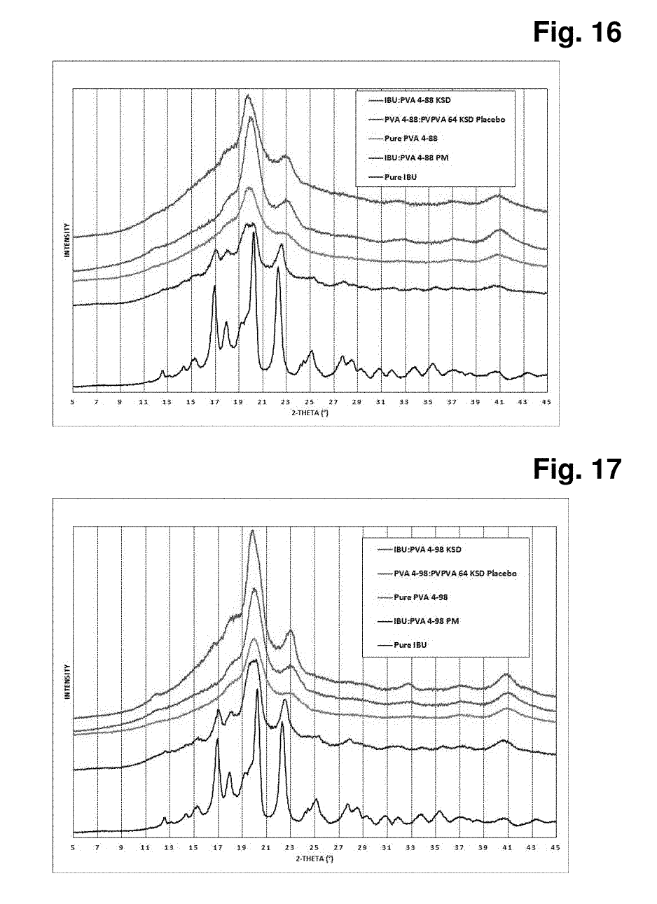

The results of XRD analysis of Composition 21 KinetiSol® product are shown in FIG. 16. It is seen in this figure that the KinetiSol® product is XRD amorphous with respect to IBU and contains crystalline character related to the polymer. A KinetiSol® processed placebo was also included in this analysis to demonstrate that the crystalline peak at 23° (2-theta) is associated with the recrystallization of PVA 4-88 after KinetiSol® processing and not related to IBU. It is also seen in this analysis that pure PVA 4-88 is moderately crystalline which substantiates the previous discussion regarding the poor processability of PVA 4-88 with IBU owing to the significant processing energy required to render the polymer molten.

FIG. 16: XRD diffractogram overlay of Composition 21 KinetiSol® (KSD) product (shown as IBU:PVA 4-88 KSD in the legend for brevity) with the corresponding physical mixture (PM), KinetiSol® processed placebo, pure PVA 4-88, and pure IBU.

The results of XRD analysis of Composition 22 KinetiSol® product are shown in FIG. 17. It is seen in this figure that the KinetiSol® product is XRD amorphous with respect to IBU and contains substantial crystalline character related to the polymer. A KinetiSol® processed placebo was also included in this analysis to demonstrate that the crystalline peak at 23° (2-theta) is associated with recrystallization of PVA 4-98 after KinetiSol® processing and not related to IBU. It is also seen by this analysis that pure PVA 4-98 is significantly crystalline which substantiates the previous discussion regarding the poor processability of PVA 4-98 with IBU owing to the significant processing energy required to render the polymer molten.

FIG. 17: XRD diffractogram overlay of Composition 22 KinetiSol® (KSD) product (shown as IBU:PVA 4-98 KSD in the legend for brevity) with the corresponding physical mixture (PM), KinetiSol® processed placebo, pure PVA 4-98, and pure IBU.

In summary, XRD analysis determined that amorphous solid dispersions of IBU with PVA 4-38, 4-75, 4-88, and 4-98 can be produced by KinetiSol processing. It was also confirmed that PVA crystallinity increases with increasing degree of hydrolysis which explains the processing issues encountered for IBU with PVA 4-88 and 4-98.

### Dissolution

A comparative dissolution analysis of the four KinetiSol® processed IBU-PVA products versus pure IBU can be revisited in Table 10. The dissolution rate of pure IBU is slow and reaches a final concentration just beyond 0.02 mg/ml after 2 hours. Composition 16 shows a somewhat faster dissolution rate versus pure IBU and reaches a final concentration of 0.043 mg/ml at 2 hours. Composition 22 shows a substantially more rapid dissolution rate versus pure IBU and Composition 16 and reaches a final concentration of 0.078 mg/ml at 2 hours. Composition 21 exhibits a more rapid dissolution rate relative to the Composition 22 yet achieves a similar final concentration of 0.077 mg/ml at 2 hours. Finally, Composition 20 exhibits a similar initial dissolution rate relative to the Composition 21, yet achieves significantly greater final concentration of 0.088 mg/ml at 2 hours.

The solubility limitations of crystalline IBU are apparent from the slow and limited dissolution of pure IBU observed from this experiment. Some benefit of converting IBU to the amorphous form is seen in the dissolution performance of Composition 16 as both the rate and extent of dissolution were improved relative to pure IBU. However, of the four KinetiSol® products, Composition 16 was the poorest performer, which is expected considering that the polymer functional groups are primarily (62%) hydrophobic acetate moieties. Composition 22 exhibited a substantial improvement in the rate and extent of IBU dissolution relative to the pure API and Composition 16; however, the dissolution rate was somewhat slower than Composition 21 and the rate and extent of dissolution were both substantially less than Composition 20. The improved dissolution performance relative to Composition 16 is due to the substantially more hydrophilic nature of the 98% hydrolyzed grade (Composition 22) versus the 38% hydrolyzed grade (Composition 16); however, the high degree of hydrolysis also imparts substantially more crystallinity onto the polymer which significantly decreases the polymer's dissolution rate, thus resulting in inferior dissolution performance relative to the PVA 4-88 (Composition 21) and 4-75 (Composition 20) based compositions. Composition 21 was also demonstrated to substantially improve the rate and extent of IBU dissolution with a 12-fold increase in IBU concentration at 30 minutes and over 3-fold increase at 2 hours relative to pure IBU. Interestingly, Composition 20 was demonstrated to provide the greatest IBU solubility/dissolution enhancement of the four KinetiSol® compositions, yielding a 14-fold increase in IBU concentration at 30 minutes and nearly a 4-fold increase at two hours relative to pure IBU. The superior performance of the PVA 4-75 based composition (Composition 20) can be attributed to the amphiphilic nature of the polymer in that it contains 25% hydrophobic acetate groups and 75% hydrophilic alcohol groups. It is likely that IBU interacts with the hydrophobic acetate moieties on the polymer to stabilize the supersaturated drug in solution while the alcohol groups provide the hydrophilicity necessary to allow for hydration and dissolution of the drug-polymer complex. This combination of hydrophobic and hydrophilic properties allows PVA 4-75 to act as a polymeric surfactant to increase the dissolution rate and solution concentrations of IBU.

1. Composition comprising one or more poorly soluble pharmaceutical active ingredient(s), which is (are) homogeneously dispersed in a polyvinyl alcohol (PVA) matrix as functional excipient, obtainable in a method, characterized in that
a) PVA, least one poorly soluble pharmaceutical active ingredient and optionally at least one processing agent are placed in a chamber of a thermokinetic mixer,
b) the substances submitted are thoroughly compounded in a thermokinetic mixer for less than 300 seconds, preferably for a duration time between 5 and 180 seconds, more preferably between 7 to 60 seconds, but most preferably between 10 to 30 seconds to minimize the heat exposure of compounded materials,
whereby the temperature in the chamber of the thermokinetic mixer is raised to 100 to 200° C. by rotational shear and friction energy, preferably to a temperature in the range of 100-150° C., in particular to a temperature in the range of 100-130° C., and
c) whereby the pharmaceutically active ingredient(s), the functional excipient and the processing agent(s) optionally submitted form a melt blended pharmaceutical composition.

2. Composition according to claim 1, wherein the comprising pharmaceutically acceptable PVA has a degree of hydrolysis in the range of greater than 72.2% but less than 90% according to the requirements of the European Pharmacopoeia or between 85-89% according to the United Stated Pharmacopoeia, and a molecular weight in the range of 14 000 g/mol to 250 000 g/mol.

3. Composition according to claim 1, wherein the poorly soluble pharmaceutical active ingredient is a biologically active agent in form of a weak base, a weak acid or a neutral molecule.

4. Composition according to claim 1, wherein the comprising pharmaceutically acceptable PVA is composed of one or more grades of PVA of differing molecular weights and of differing grades of hydrolysis.

5. Composition according to claim 1, wherein the comprising pharmaceutically acceptable PVA is combined with another excipient.

6. Composition according to claim 5, wherein PVA as functional excipient is combined with another pharmaceutically acceptable polymer.

7. Composition according to claim 1, comprising a week base as biologically active agent and PVA in a ratio in the range of 1:99 to 1:1 by weight, preferably the ratio of active agent to PVA is in the range 1:70 to 1:2.

8. Composition according to claim 1, wherein the comprising active agent is ground or pre-milled to mean particle sizes in the range of 1 to 1000 μm, preferably to mean particle sizes in the range of 1 μm to 100 μm, most preferably in the range of 10 μm to 100 μm, before it is processed.

9. Composition according to claim 1, comprising the pharmaceutical active ingredient(s) in an amorphous nano-crystalline or micro-crystalline form.

10. Composition according to claim 1, in which the pharmaceutical active ingredient, upon dissolution, is dissolved by a factor of at least 1.2 higher compared to the thermodynamic solubility of said ingredient alone in the polymer matrix.

11. Composition according to claim 1, wherein the comprising PVA is crystalline, semi-crystalline or amorphous after processing.

12. Composition according to claim 1, wherein the poorly soluble pharmaceutical active ingredient is selected from the group itraconazole, ibuprofen and nifedipine.

13. Composition according to claim 1, comprising itraconazole in a amorphous solid dispersion wherein itraconazole and pharmaceutically acceptable polyvinyl alcohol (PVA), preferably PVA 4-88, are present in a weight ratio in the range from 1:99 to 1:1, preferably in a weight ratio of itraconazole to PVA in the range from 1:70 to 1:2.

14. Composition according to claim 1, comprising nifedipine in a amorphous solid dispersion wherein nifedipine and pharmaceutically acceptable polyvinyl alcohol (PVA), preferably PVA 4-88, are present in a weight ratio in the range from 1:99 to 1:1, preferably in a weight ratio of nifedipine to PVA in the range from 1:70 to 1:2.

15. Composition according to claim 1, comprising ibuprofen in a amorphous solid dispersion wherein ibuprofen and pharmaceutically acceptable polyvinyl alcohol (PVA), preferably PVA 4-75, are present in a weight ratio in the range from 1:99 to 1:1, preferably in a weight ratio of ibuprofen to PVA in the range from 1:70 to 1:2.

16. Oral dosage form comprising a composition according to claim 1 in form of tablets, beads, granules, pellets, capsules, suspensions, emulsions, gels, films.
